(19) United States
(12) Patent Application Publication
(10) Pub. No.: US 2015/0164327 A1
(43) Pub. Date: Jun. 18, 2015

(54) MULTIMODAL IMAGING FOR THE DETECTION OF TISSUE STRUCTURE AND COMPOSITION

(71) Applicant: University of Massachusetts, Boston, MA (US)

(72) Inventors: Anna N. Yaroslavsky, North Andover, MA (US); Robert H. Giles, Lowell, MA (US); Cecil S. Joseph, Lowell, MA (US); Rakesh Patel, Sharon, MA (US)

(73) Assignee: UNIVERSITY OF MASSACHUSETTS, Boston, MA (US)

(21) Appl. No.: 14/414,675

(22) PCT Filed: Jul. 15, 2013
(86) PCT No.: PCT/US2013/050535

§ 371 Date: Jan. 13, 2015

Related U.S. Application Data
(60) Provisional application No. 61/671,540, filed on Jul. 13, 2012.

(51) Int. Cl. A61B 5/00; A61B 5/05

(52) U.S. Cl. CPC A61B 5/0035; A61B 5/0507; A61B 5/7278; A61B 5/0064; A61B 5/7246; A61B 5/4872; A61B 5/443; A61B 5/444; A61B 5/7282; A61B 5/004; A61B 2576/02

(57) ABSTRACT

The present invention relates to the use of optical and terahertz imaging of tissue for measuring characteristics to assist in diagnosis. A light delivery and collection system is used that can aid in the detection of tumor margins, for example. A data processor processes the image data to determine characteristics of a region of tissue.

### CROSS REFERENCE TO RELATED APPLICATIONS

This application claims priority to U.S. Application No. 61/671,540 filed Jul. 13, 2012, the entire contents of which are incorporated herein by reference.

### BACKGROUND OF THE INVENTION

With approximately 3.5 million cases diagnosed each year, nonmelanoma skin cancer (NMSC) is the most common form of cancer. NMSC results in about 3000 deaths each year and the cost of treatments is estimated to exceed $600 million each year. The most effective form of NMSC treatment is Mohs Micrographic Surgery (MMS) which involves removing cancer layer by layer while simultaneously processing excised tissue for frozen H&E histopathology to map out the cancerous regions. MMS has a success rate of 95% and is the only technique that examines entire surgical margin allowing for complete histological assessment during surgery. However MMS is time consuming, labor intensive and costly.

Continuous wave terahertz imaging (CWT) is used to differentiate between nonmelanoma cancers and normal skin. The terahertz region of electromagnetic spectrum extends from 30 μm to 3000 μm (10 THz to 0.1 THz) and lies between the microwave and infrared regions. Terahertz radiation is non-ionizing and medical applications of this frequency region are being explored. There is a difference in bound and free water content between normal and cancerous tissue.

Contrast between cancerous and normal tissue can be obtained using a continuous wave terahertz system. One of the disadvantages of terahertz imaging for biomedical applications is the inherent lack of resolution, which prevents terahertz radiation from identifying tissue morphology.

Polarized-light imaging is an optical technique that is capable of obtaining superficial images of thick tissue layers. When the light incident on the sample is linearly polarized, subtraction of two images acquired with the co-polarized (I_{∥}) and cross-polarized (I_{⊥}) light can be used to isolate the single-scattered component, which arises mainly from superficial skin layers. The advantages of the polarized light imaging include the ability to image comparatively thin tissue layers (˜30 μm-200 μm in the 380 nm-750 nm spectral range) and to retain a large field of view. Optical images can be acquired within milliseconds. The combination of the large field-of-view, rapid image acquisition, and sufficient lateral resolution enables rapid examination of large surfaces, thus facilitating tumor margin delineation. Dye-enhanced multi-spectral reflectance imaging enables reliable delineation of cancerous and normal tissue in more than 91% of cases. However, white light polarization imaging and intrinsic contrast polarization imaging fail to provide sufficiently high resolution and cancer contrast, respectively.

Intrinsic optical imaging yields high resolution, but often lacks contrast for reliable detection of cancer. Terahertz imaging detects intrinsic contrast between healthy and cancerous tissue, but has low resolution for the measure amount of tissue morphology. Thus, further improvement are needed to existing system and method for cancer margin delineation.

### SUMMARY OF THE INVENTION

The present invention utilizes optical and terahertz imaging for accurate nonmelanoma skin cancer (NMSC) delineation. An illumination and light collection system is used to deliver light from a light source system onto a tissue region to be measured. In a preferred embodiment, the light source system can include a first source emitting in the terahertz region of the electromagnetic spectrum and a second source emitting in the optical region of the electromagnetic spectrum. A detection system can include a first detector that detects light in the terahertz region of the spectrum and a second detector detection optical wavelength. A data processor receives image data from both detectors and determines characteristics of the tissue based on the detected image data. Both morphological information and molecular composition of the tissue can be analyzed and determined.

Terahertz reflectance of NMSC can be quantified to demonstrate that cross-polarized terahertz images can correctly identify the location of tumors. Cross-polarized and polarization difference optical images can accurately present morphological features. Cross-polarized terahertz images exhibited lower reflectivity values in cancer as compared to normal tissue, for example, and can thus provide diagnostically useful information.

A preferred embodiment of the invention provides a support on which a tissue sample can be positioned to enable imaging without movement of the sample. A scanning system can be employed to scan the tissue with a beam of light. The system can deliver continuous wave terahertz wavelengths onto the tissue. The detected image data can be processed with a data processor that is programmed to process both the optical image data and the terahertz image data to detect characteristics of the tissue such as the size and shape of cancerous lesions or tumors.

Another preferred embodiment provides a portable system for clinical use. This system enables illumination of the sample by both terahertz source and an optical source without moving the sample.

### BRIEF DESCRIPTION OF THE DRAWINGS

FIG. 1A is a schematic illustration of terahertz reflection system;

FIG. 1B is a schematic illustration of a terahertz transmission system in accordance with the invention;

FIG. 2 is a schematic of polarized light imager in accordance with the invention;

FIG. 3A-3E show specimen with infiltrative BCC, (a) shows the co-polarized terahertz reflectance image, (b) shows the cross-polarized terahertz reflectance image, (c) shows the H&E stained histology of a 5 μm frozen section of the tissue, (d) shows the cross-polarized optical image, and (e) shows the polarized light image;

FIGS. 4A-4I show comparison of magnified high resolution optical and histology images of morphological features, specified from outlined boxes in the sample histology of the infiltrative BCC specimen (FIG. 3c);

FIGS. 5A-5E illustrate a specimen with SCC, (A) shows the co-polarized terahertz reflectance image, (B) shows the cross-polarized terahertz reflectance image, (C) shows the H&E stained histology of a 5 μm frozen section of the tissue, (D) shows the cross-polarized optical image, and (E) shows the polarized light image;

FIGS. 6A-6L show a comparison of magnified high resolution optical and histology images of morphological features specified from outlined boxes in the sample histology of the SCC specimen (FIG. 5C);

FIG. 7 graphically illustrates normal and cancer mean terahertz reflectivity values (%), averaged over all BCC, SCC, and total samples;

FIG. 8 illustrates an optical imaging system in accordance with a preferred embodiment of the invention;

FIG. 9 illustrates a terahertz transceiver in accordance with a preferred embodiment of the invention;

FIG. 10 illustrates a terahertz scanning system that can be used in conjunction with preferred embodiments of the invention;

FIG. 11A illustrates a terahertz and optical imaging system;

FIG. 11B illustrates an off-axis imaging system in accordance with the invention;

FIG. 11C illustrates use of a scanning terahertz imaging system in combination with an optical imaging system in accordance with preferred embodiments of the invention;

FIG. 12 shows a process sequence for measuring and analyzing tissue in accordance with the invention;

FIGS. 13A-13C show images of a tissue sample using cross polarized imaging at 450 nm, reflectance polarization at 450 nm and a standard histology image, respectively;

FIGS. 14A-14G illustrate comparative imaging analysis of the methods described herein;

FIGS. 15A and 15B illustrate terahertz imaging and a standard histology image;

FIGS. 16A and 16B compare cross-polarized and reflectance images as described herein;

FIGS. 17A and 17B compare cross-polarized images and histology images as described herein;

FIGS. 18A-18I illustrate detailed features of optical cross-polarized images, terahertz processing of images and standards histology;

FIGS. 19A-19G illustrate detailed features of cancerous and normal regions as described herein; and

FIG. 20 shows a method of terahertz processing as described herein.

### DETAILED DESCRIPTION OF THE INVENTION

The present invention relates to systems and methods for measuring tissue that uses terahertz imaging of tissue to provide diagnostic information. Details regarding the use of continuous wave transmission imaging are described in more detail by Joseph et al., “Continuous Wave Terahertz Transmission Imaging of non-melanoma Skin Cancer,” Lasers in surgery and Medicine, 43: 457-462(2011), the entire contents of which is incorporated herein by reference.

For the measurements an optically pumped far-infrared (FIR) gas laser (carbon dioxide). The output power of such a laser can be in the range of 100-150 W, for example. Tuning the output frequency of the laser allows the pumping of different transitions of the gas in the FIR cell. Selecting the gas in the FIR cell and the tuning of the laser to the appropriate pump frequency provides the ability to lase different frequencies in the terahertz region. A 584 GHz (513 μm) vertically polarized transition in HCOOH is used, pumped by the 9R28 transition of the laser. The measured output power was 10.23 mW. A dielectric (glass) waveguide was placed at the output of the FIR lasers to obtain a Gaussian beam profile. A liquid helium cooled silicon bolometer manufactured by IRLabs was used as a detector. The noise equivalent power (NEP) of the detector was 1.13×10⁻¹³ W/√Hz and the responsivity was 2.75×10⁺⁵ V/W. The bolometer had a response time of 5 ms and the gain was 200. A Garnet powdered crystalline quartz window on the bolometer rejected wavelengths below 100 μm.

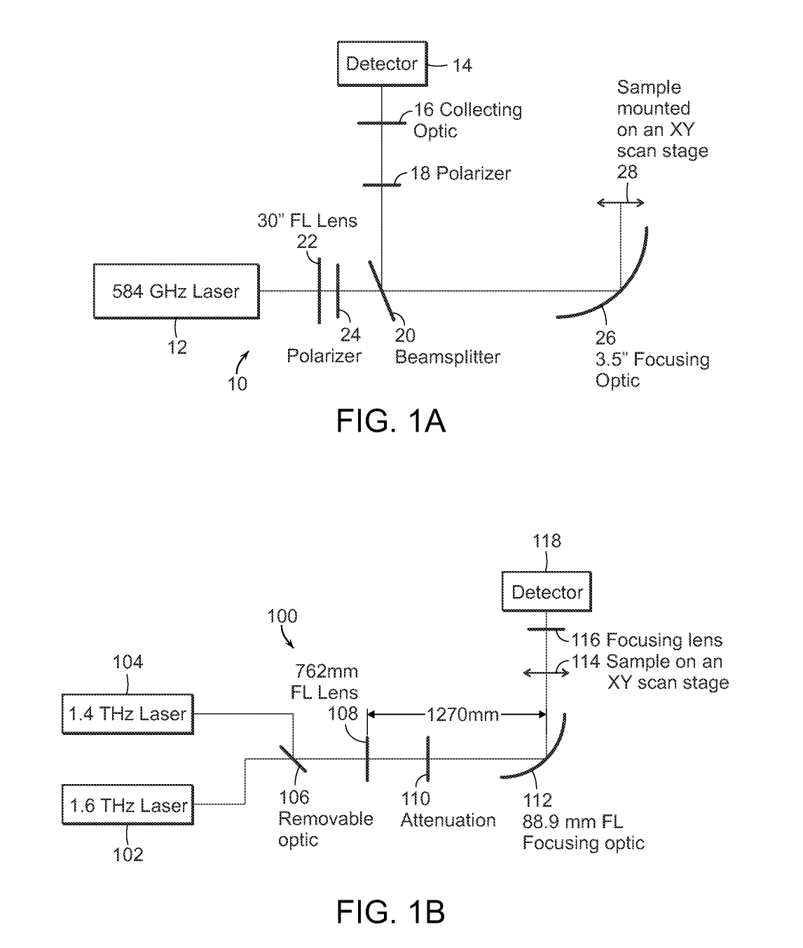

FIG. 1A shows a schematic of the measurement system 10. The laser beam from laser 12 was collimated using a TPX lens 22 passed through a wire grid polarizer 24 to clean up the polarization of the transmitted beam, and focused onto sample mounted on a scanning stage 28 in which the imaging plane is positioned using a short focal length off axis parabolic mirror 26. The full width at half max (FWHM) was measured to be 0.67 mm at the sample plane. The signal remitted from the sample goes back through the system and, after the focusing mirror, is redirected into the detector arm by a 50-50 Mylar beam splitter 20. The signal is then passed through an analysing wire grid polarizer 18, which can be oriented to transmit either co-polarized or cross-polarized light and collected using an off axis parabolic mirror 16 and focused into a detector 14 such as a bolometer. An automated two-axis stage was used to raster scan the sample in the imaging plane. The scanning resolution of both the horizontal and vertical axes was set to 0.1 mm. The laser beam was optically modulated. The modulating frequency served as the reference for a lock-in amplifier. Data acquisition times for the images collected were determined by the speeds of the translation axes used for this experiment and the size of the samples. The dwell time per point in the image was around 150 ms and the system signal-to-noise ratio (SNR) using a lock-in amplifier was 65 dB.

A system 100 for transmission imaging in accordance with preferred embodiments is illustrated in FIG. 1B. In this embodiment the sample is positioned on a computer controlled scanning stage 114 and light from the light source system including lasers 102 and 104 is transmitted using a moveable optical switch 106, a lens 108, an attenuator 110, and a focusing optical element 112, through the sample and a focusing lens 116 directs the transmitted light onto the detector 118.

Terahertz images were processed using a Labview™ program that synchronized the sample position in the imaging plane with the return signal from the lock-in amplifier. Co-polarized and cross-polarized images were acquired by selecting the appropriate orientation with the analysing polarizer in the reflectance arm of the system. They were then calibrated against the full-scale return from a flat front surface gold mirror to determine the reflectance. The off sample areas were removed in post-processing and the image was plotted in logarithmic space. The reflected terahertz signal measured from each specimen was quantified pixel by pixel using the formula 1:

$${R}_{THz} = \frac{{I}_{measured}}{{I}_{incident}} \times 100 \% \qquad ( 1 )$$

where R_{THz} is the terahertz reflectance value in percent (%) I_{measured} is the measured reflectance intensity from the sample, and I_{incident} is the measured intensity of the incident beam reflected from a gold front-surface flat mirror.

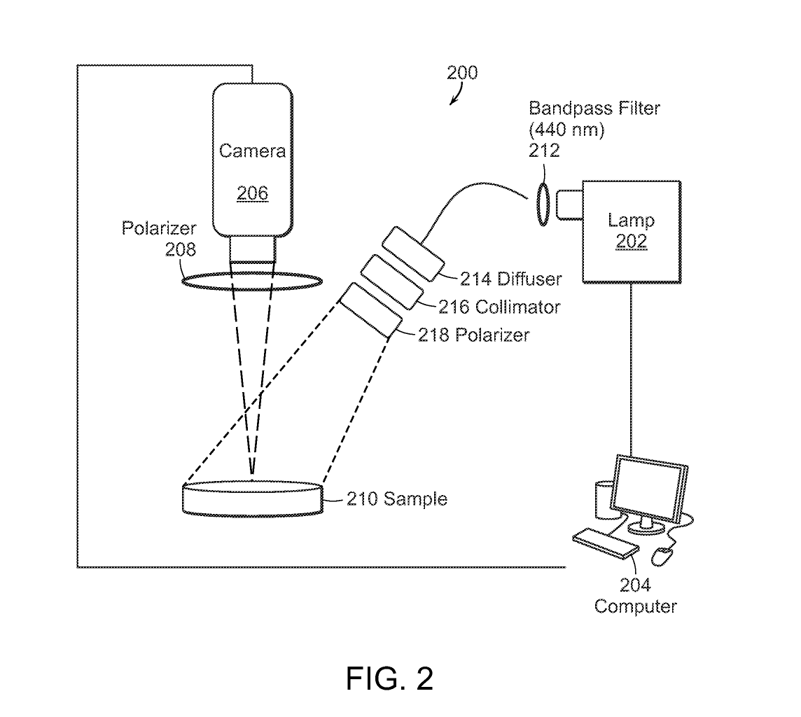

Depicted in FIG. 2, the optical imaging system 200 used to acquire reflectance and PLI images of the sample consists of a xenon arc lamp 202 (Lambda LS, Sutter, Novanto, Calif.) combined with a narrow bandpass filter 212 (full width at half max: 10 nm) as well as a diffuser 214 and collimator 216 to provide monochromatic light at 440 nm. The optical image was detected by a detector 206 such as a CCD camera (CoolSnap Monochrome Photometrics, Roper Scientific, Tucson, Ariz.) with an attached 0.5 Rodenstock lens (Linos Photonics, Qioptiq, Luxembourg) resulting in a large field of view (2.8 cm×2.5 cm). Linear polarizing filters 218, 208 (Meadowlark Optics, Frederick, Colo.) were positioned in the beam path incident on and remitted from the sample 210. These filters allowed collection of both co-polarized and cross-polarized reflection images by orienting the analysing polarizer parallel (co-) or perpendicular (cross-) to the polarization of the incident light. Polarization images were processed by applying equation 2, where PLI is the polarization light image, I_{co} is the co-polarized image, I_{cross} is the cross-polarized image, and G is a calibration factor which accounts for any bias in the system towards either polarization.

PLI=I_{∞}−G×I_{cross} (2)

The system provided rapid automatic image acquisition (total acquisition time was <100 ms) controlled through Metamorph software (Molecular Devices, Inc., Sunnyvale, Calif.). The lateral resolution was measured to be better than 15 μm. The calibration factor, G, of the system as described was measured to be 0.98. The computer or data processor 204 can be connected the light source, detector and other system components as described herein to operate the system and processes image data.

Fresh thick excess cancer specimens were obtained within 2 hours after Mohs micrographic surgeries. The samples were imaged within 6 hours. For imaging, the specimens were covered with a 1 mm thick z-cut quartz window. To prevent dehydration during the measurement, the samples were placed on a gauze soaked in pH balanced (pH 7.4) saline solution. En face frozen hematoxylin and eosin (H&E) sections were processed from the imaged specimens. These frozen H&E sections were used as a standard for evaluation of the results yielded by optical and terahertz images.

The correlation of optical and terahertz images to histopathology was performed. Due to preparation of frozen H&E sections, processed histology sections were slightly distorted in comparison to size and shape of the real samples. This can cause discrepancies in correlation of the optical and terahertz sample images to histology. To facilitate a more accurate analysis, the histological slides were digitized and identified four to ten pairs of common features in histology and in the optical images. Then optical and histopathological images were overlaid by applying affine transformations. Normal and cancer areas were demarcated by a pathologist on the digitized histology image. Corresponding normal and cancer areas were projected from the digitized histology onto the optical and terahertz images.

Reflectance values obtained from terahertz images were averaged for representative normal or cancer areas of each sample. The reflectance values corresponding to cancer and normal respective regions obtained for each sample were averaged over all basal cell carcinomas (BCC), squamous cell carcinomas (SCC), and all the samples (BCC+SCC). To quantify the significance of the differences between normal and cancerous tissue, the data was evaluated using a 1 tailed student's t-test for 2 independent populations. The significance test was performed on the mean values averaged over all samples imaged. P-values were reported to indicate the significance of the differences.

In total, 9 specimens from 9 subjects, which included 6 basal cell carcinomas (BCC) and 3 squamous cell carcinomas (SCC). The final diagnoses were based on the analysis of the frozen H&E histopathology processed during the micrographic surgeries. The information on the imaged specimens is summarized in Table 1 columns 1-4.

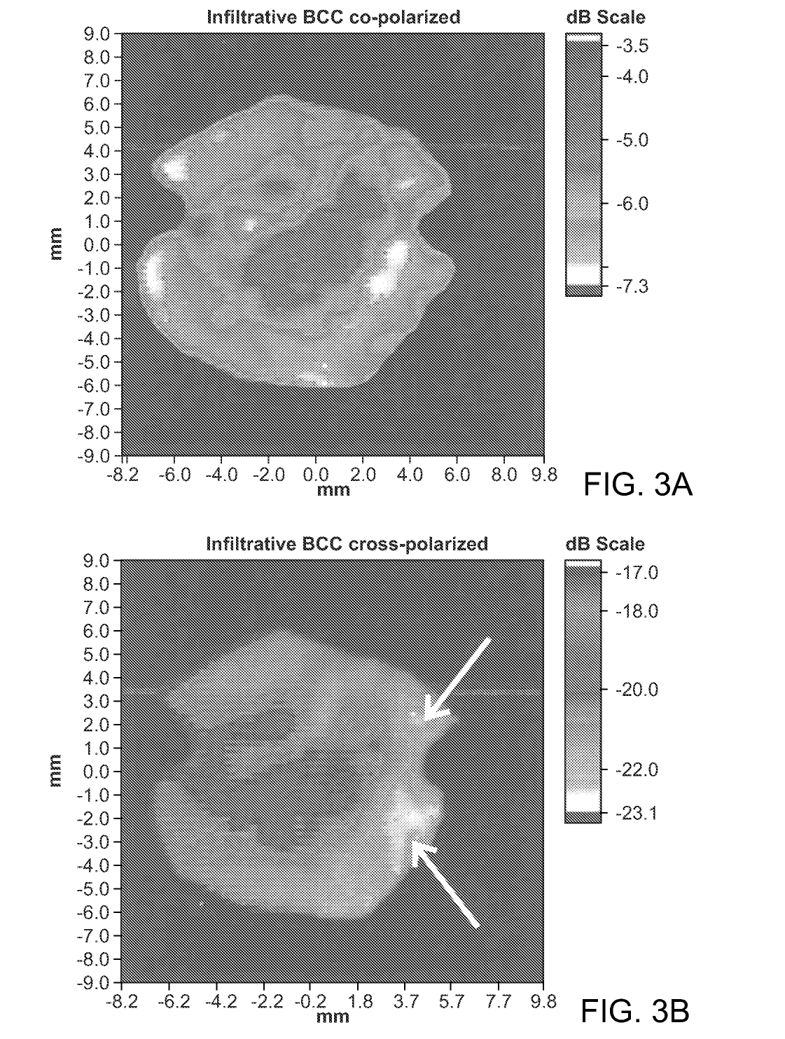

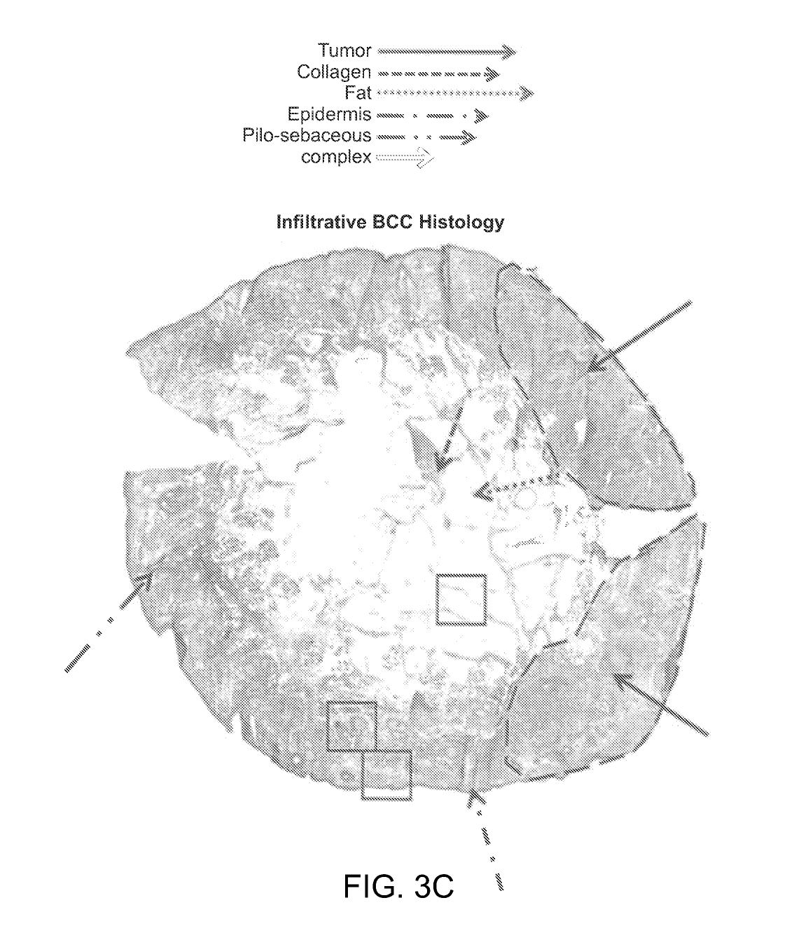

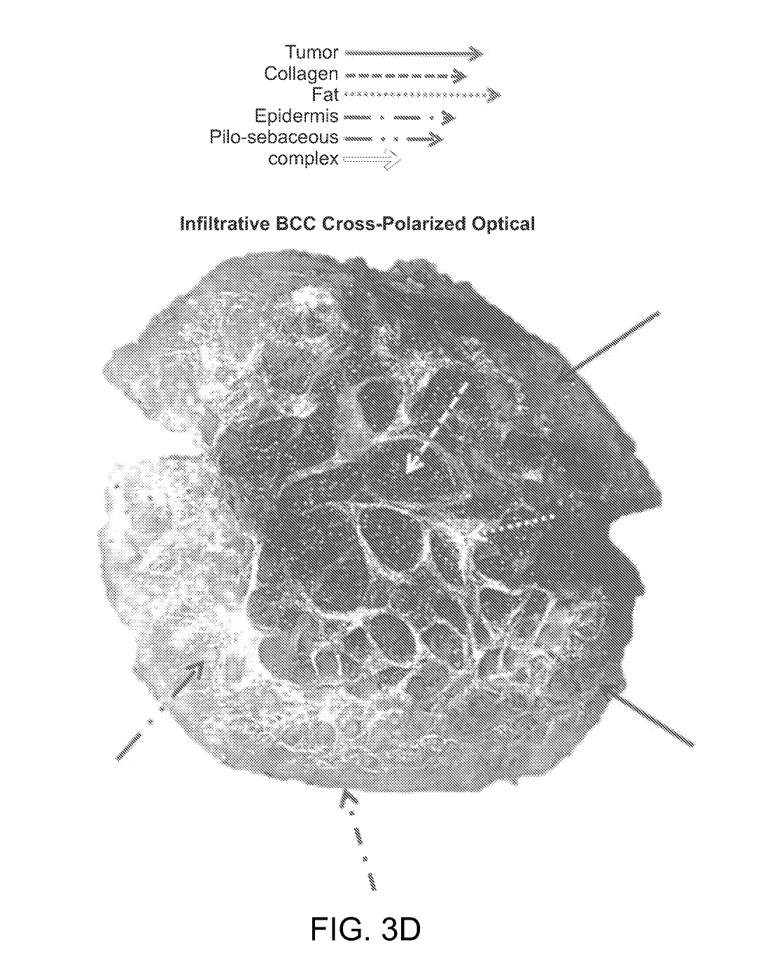

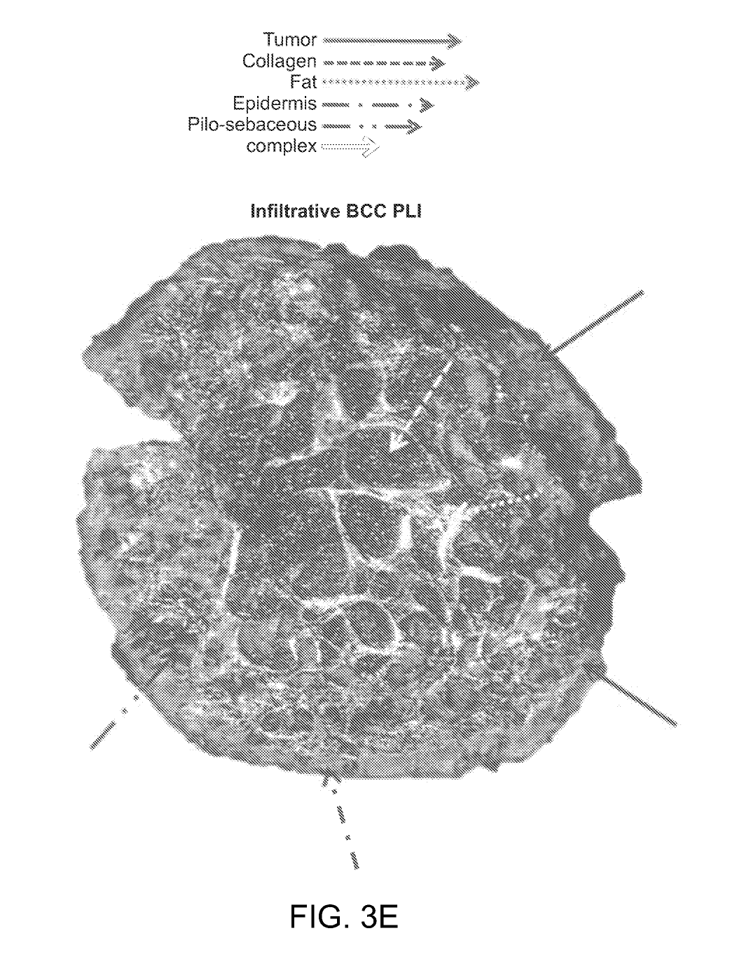

A representative sample of basal cell carcinoma is shown in FIG. 3A-3E. BCC is the most common skin cancer type. It rarely metastasizes. However, because it can cause significant destruction, disfigurement and morbidity by invading surrounding tissues, it is still considered malignant. In FIGS. 3A and 3B, the terahertz co-polarized and cross-polarized images are presented. The tumour is outlined with the black dotted line in the H&E histopathology (FIG. 3C). It can be appreciated that the tumour region correlates well with the size and shape of low reflectance areas in the cross-polarized terahertz image (FIG. 3B). The location of the tumour is indicated by a solid arrow in the terahertz cross-polarized image (FIG. 3B). The co-polarized terahertz image (FIG. 3A) does not correlate with the sample histology as well as cross-polarized image. In particular, the areas of lower reflectivity don't correlate with cancer affected area histopathology. The difference in the appearance of the co- and cross-polarized terahertz images is primarily due to specular reflection of the air cover glass and cover glass tissue interfaces, which contribute to the co-polarized image. The majority of Fresnel signal comes from the reflection of the incident radiation on the glass air interface and, therefore, does not contain information on the sample. However, it cannot be rejected from the co-polarized terahertz image due to the geometry of the experiment (FIG. 1A). In contrast, imaging cross-polarized terahertz signal enables effective removal of the specular reflections, as the Fresnel component is co-polarized with the incident radiation.

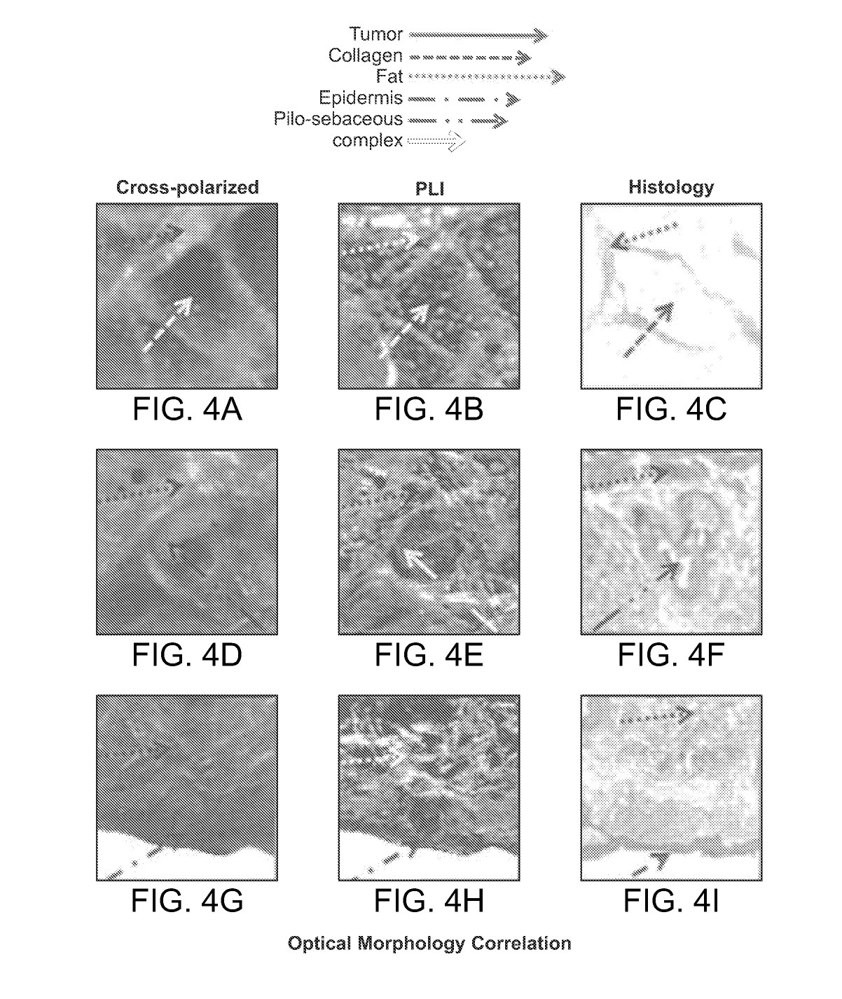

The optical reflectance cross-polarized and polarization difference images are presented in FIGS. 3D and 3E, respectively. As compared to terahertz images (FIGS. 3A-3B), optical images of the same specimen offer higher resolution. The comparison of optical images (FIGS. 3D-3E) to histology (FIG. 3C) reveals a close correlation of morphological features as well as overall size and shape of the sample. Polarization difference imaging in skin at 440 nm enables optical sectioning to about 50-70 μm. However, the polarization difference image often provides lower contrast as compared to the cross-polarized image. Therefore, both images were used for tissue morphology analysis. To demonstrate the resolution afforded by optical imaging, the magnified section of regions outlined in histology (FIG. 3C: boxes) are presented in FIGS. 4A-4I. The optical images clearly show morphological features such as the epidermis (dash-dot arrow), pilo-sebaceous complex (dash-dot-dot arrow), subcutaneous fat (dot-dot arrow), as well as highly reflective collagen strands (dash-dash arrow). The tumour region (solid arrow), characterized by the loss of skin appendages and collagen appears as a homogenous dark area as seen in FIGS. 3D and 3E.

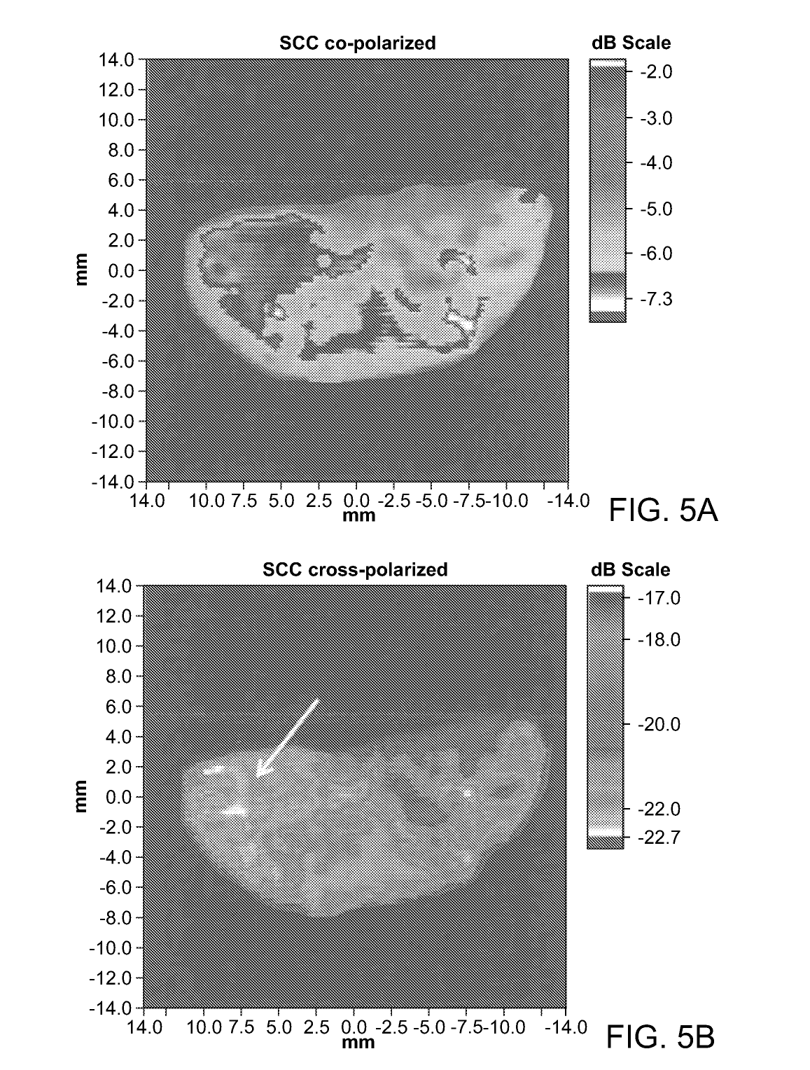

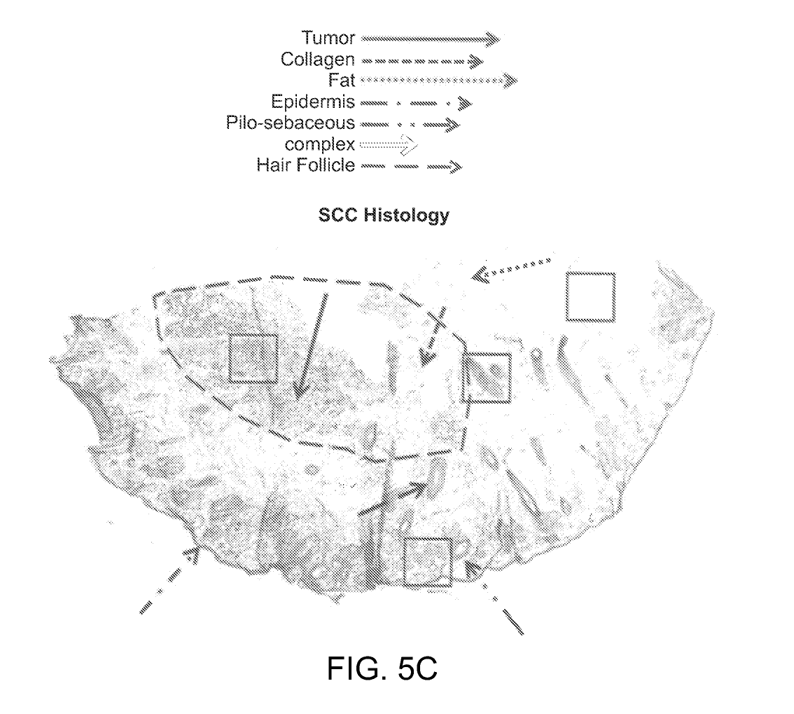

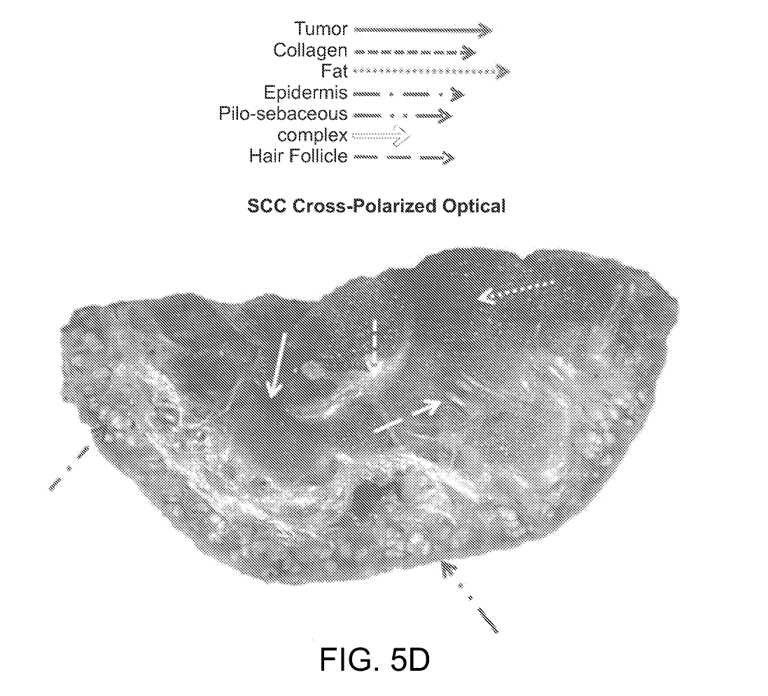

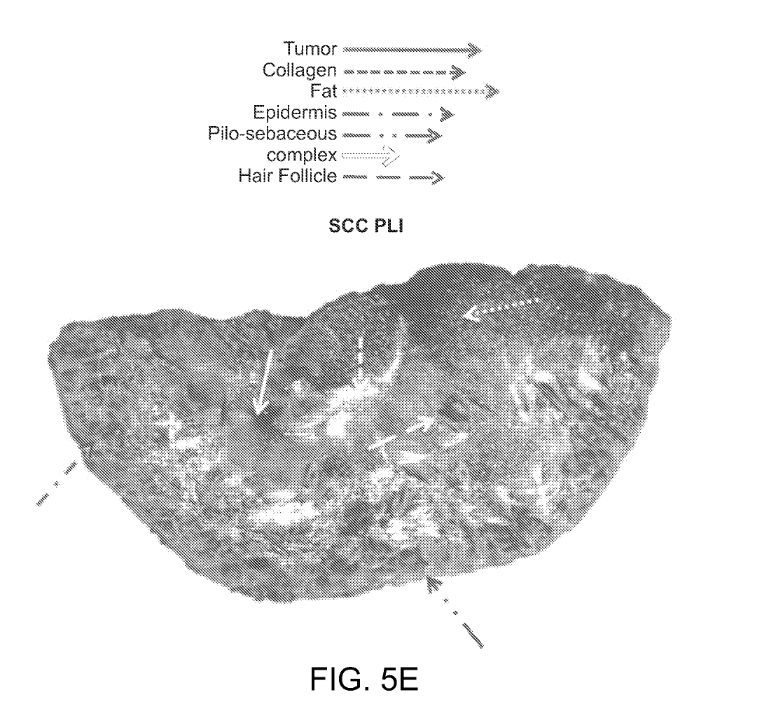

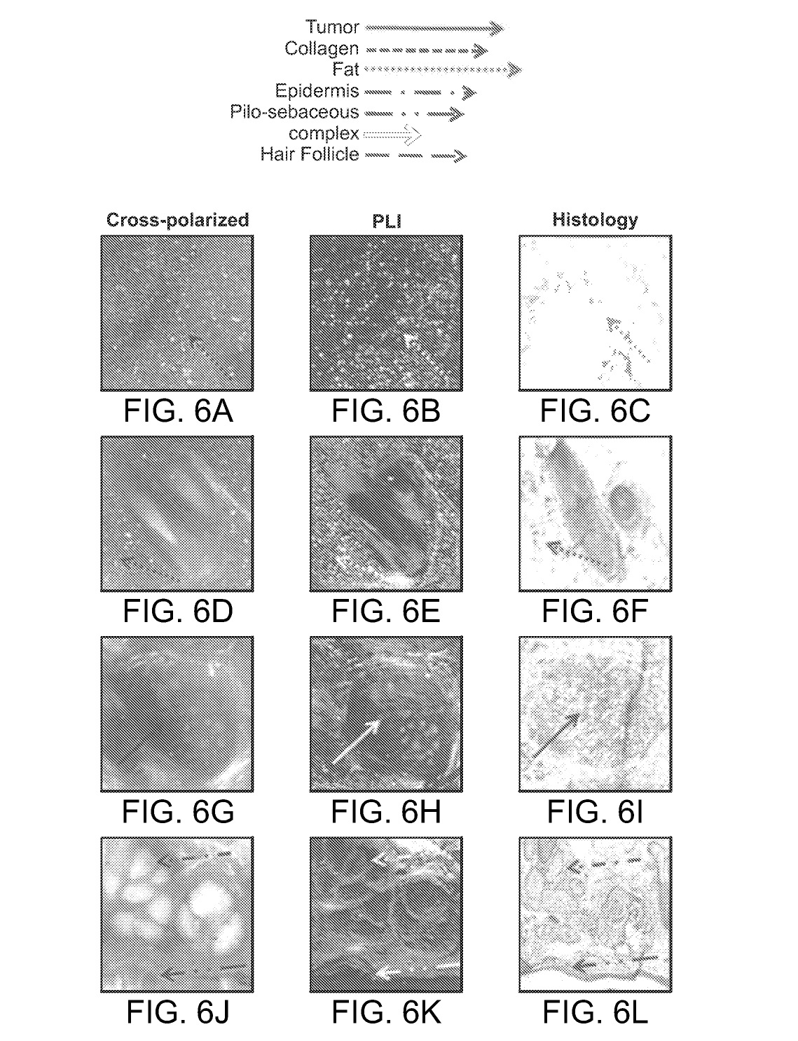

FIGS. 5A-5E show a representative specimen with squamous cell carcinoma. While only 20% of nonmelanoma cancers are squamous cell carcinomas, they tend to be more aggressive than basal cell cancers. They are more likely to invade fatty tissues beneath the skin and, although this is still uncommon, spread to lymph nodes and/or distant parts of the body. Comparison of the co- and cross-polarized terahertz images (FIGS. 5A-5B) with H&E histopathology presented in FIG. 5C, demonstrates that the cross-polarized terahertz image correctly highlights the location of cancer (solid arrow) as in the case of BCC. Similarly, comparison of the cross-polarized terahertz image (FIG. 5B) with the cross-polarized reflectance and the superficial optical images shown in FIGS. 5D and 5E, respectively, confirms that the tumour area shows up dark in the optical images, indicating a lack of collagen and loss of structure. Comparison with histology (FIG. 5C: tumor outlined with dashed line) shows that terahertz images (FIGS. 5A-5B) have higher contrast of the tumour whereas optical images (FIGS. 5D and 5E) delineate tumor affected areas more accurately. Tumour margins, as well as other skin appendages are clearly visible in the optical images. In FIGS. 6A-6L, higher magnification optical images of adipose tissue (FIGS. 6A-6C), hair follicles (FIGS. 6D-6F), tumor lobule (FIGS. 6G-6I), and sebaceous glands (FIGS. 6J-6L) are compared to respective structures in the H&E histopathology image. The resemblance in the appearance of optical and histological images can be well appreciated. Comparison of the optical and terahertz images, presented in FIGS. 5A, 5B, 5D and 5E, respectively, demonstrates the higher resolution offered by optical imaging.

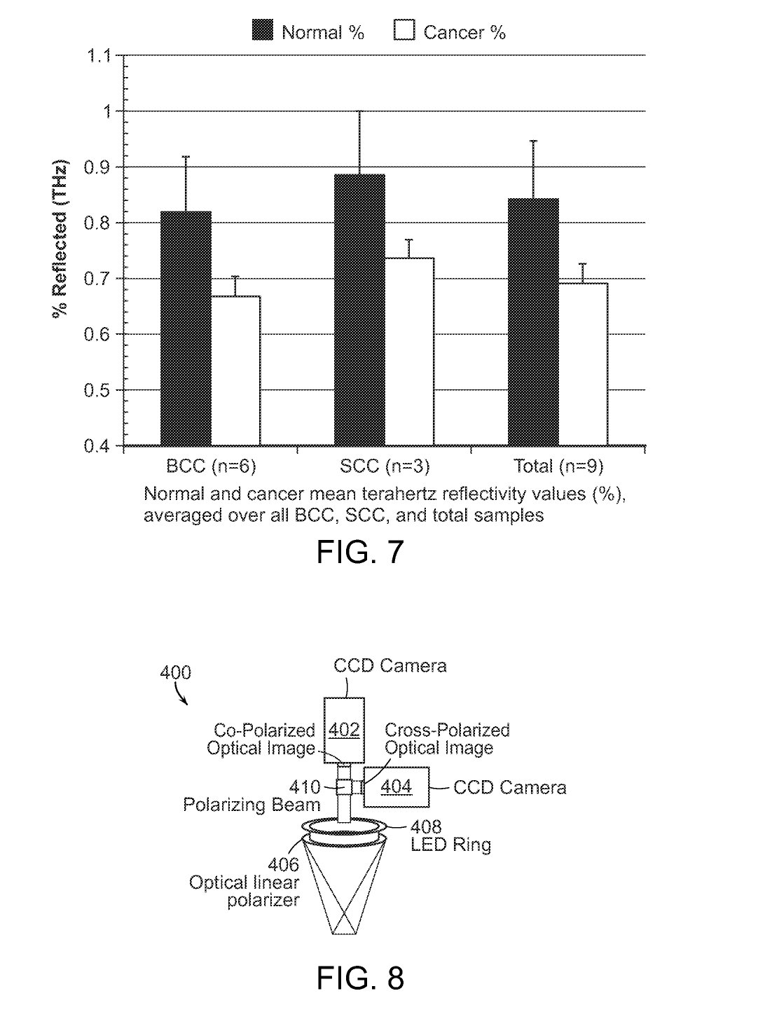

For the terahertz images, the percentage cross polarized reflectivity of representative cancerous areas was compared with the percentage cross polarized reflectivity of representative normal areas on the same sample. Table 1 summarizes the results for each specimen and histograms for the averaged data for BCC, SCC, and total samples are presented in FIG. 7.

TABLE 1

| Sample | | | | Normal | Standard | Cancer | Standard |
|---|---|---|---|---|---|---|---|
| Number | Diagnosis | Gender | Age | % reflectance (THz) | Deviation | % reflectance (THz) | Deviation: |
| 1 | BCC | Male | 76 | 0.86 | ±0.13 | 0.61 | ±0.071 |
| 2 | BCC | Female | 87 | 0.92 | ±0.12 | 0.80 | ±0.057 |
| 3 | BCC | Male | 55 | 0.65 | ±0.069 | 0.56 | ±0.013 |
| 4 | BCC | Male | 88 | 0.85 | ±0.11 | 0.71 | ±0.023 |
| 5 | BCC | Male | 60 | 0.84 | ±0.083 | 0.73 | ±0.014 |
| 6 | BCC | Male | 39 | 0.77 | ±0.071 | 0.58 | ±0.030 |
| 7 | SCC | Male | 75 | 0.74 | ±0.12 | 0.58 | ±0.018 |
| 8 | SCC | Female | 94 | 0.86 | ±0.11 | 0.77 | ±0.046 |
| 9 | SCC | Male | 66 | 1.06 | ±0.12 | 0.86 | ±0.031 |

The average reflectivity values for BCC showed that cancer had lower reflectivity than normal tissue. Similarly, SCC specimens showed the same trend but the reflectivity values were slightly higher than those for BCC samples. This can result from the low number of SCC specimens (n=3) imaged so it is difficult to draw general conclusions from this data. Overall the average cross polarized percentage reflectivity of the tumour and normal regions for all 9 samples was found to be 0.69%±0.034% and 0.84%±0.010%, respectively. The difference between normal and cancer for representative sections averaged over all samples was significant (p<0.001). These results show that even though some differences in the terahertz reflectivity values are expected across the samples, there are common threshold value for cancer and normal skin. Nonetheless, as the specimens come from patients of different ages, genders, and tumour sites these will result in different appropriate threshold values.

In the terahertz images shown in FIGS. 3A and 3B, one can see that the cross-polarized image correlates better with the sample histology (FIG. 3C). This was true for all specimens measured. Although the terahertz images indicate the approximate location of the tumor, they do not accurately demarcate the size and shape of the tumor. On the other hand, optical images provide the morphological detail necessary to outline the extent of the tumor boundaries but lack the level of contrast displayed in terahertz images. As a result, terahertz imaging may be used to detect approximate location of tumour nodule and thus guide inspection of the tumour boundaries in optical images. Having accurate tumour boundaries is crucial to ensure full resection of the tumour while preserving as much healthy tissue as possible, especially when the tumour resides on the face.

Another effect that is apparent from the terahertz data is that the cancerous region has a lower reflectivity than the noncancerous region (FIG. 7). Interestingly, this observation is similar to what is detected in optical imaging; where tumour affected areas appear darker than normal areas. There are two possible explanations for this phenomenon. Firstly, due to bound water content, cancer exhibits higher absorption relative to normal skin and therefore leads to lower remitted signal and consequently lower reflectivity of cancerous areas. Secondly, nonmelanoma cancers are defined by their loss of normal skin architecture and, given the wavelength of terahertz imaging cancerous skin can look fairly homogenous with minimal refractive index mismatch within the tumour. In contrast, normal skin has multiple structures (hair follicle, sebaceous gland, adipose tissue, epidermis, etc.) which can cause a greater local refractive index mismatch resulting in higher reflectivity.

Thus, the present invention polarization sensitive terahertz imaging for biomedical applications. By implementing cross-polarized reflectance terahertz interrogation, the present invention enables the measurement of accurate images of skin cancer tissue due to rejecting Fresnel reflections that inevitably contaminate the co-polarized component of reflected light. The results presented in FIGS. 3A-3E demonstrate that in some cases specular reflections significantly alter the appearance of the co-polarized tissue image (FIG. 3A) making delineation of BCC unattainable. In contrast, cross-polarized image of the same tissue (FIG. 3B) accurately demarcates cancer as confirmed by histopathology (FIG. 3C).

Another solution to rejecting the Fresnel component in terahertz imaging is to illuminate the imaged object at an oblique angle, similar to the optical configuration (FIG. 2). In that case, the Fresnel component is not be registered by the detector and both co- and cross-polarized component can be used for accurate imaging. Use of the oblique illumination in the terahertz spectral range it will almost double the acquired terahertz signal.

The present invention provides a combination of polarization sensitive optical and terahertz imaging provides complementary information and can be used for intraoperative delineation of nonmelanoma skin cancers. Cross-polarized terahertz imaging correctly detects the location of cancer thus guiding higher resolution optical imaging, which is capable of accessing tissue morphology on a microscopic scale and accurately delineating tumor margins. This has shown that cross-polarized terahertz reflectivity values are lower for cancerous areas with respect to normal areas. This is a step in determining threshold values for accurate detection of nonmelanoma skin cancer using terahertz interrogation. A combined system, uses algorithms for delineating tumor margins, creating fused optical-terahertz images, in the combined system.

A preferred embodiment includes a the polarization sensitive optical imager. The hardware and the software provide integration with the terahertz imager for in vivo imaging. The schematic of the optical imager 400 is presented in FIG. 8. Homogenous oblique illumination is provided by a ring 408 of light emitting diodes (LED) combined with a high contrast (1000:1) and high transmission (70%-85%) linearly polarizing filter 406. Illumination wavelengths between 395 nm and 475 nm can be used. Axial resolution of polarization difference imaging improves with decreasing wavelength. However, the wavelength of 395 nm is closer to the maximum of the Soret absorption band of hemoglobin. Thus it can be strongly affected by the presence of blood in the surgical field during in vivo imaging as compared to 475 nm. Tissue phantoms containing hemoglobin are used to calibrate the optical imager and enable selection of illumination wavelength with respect to contrast, resolution, and acquisition time.

To enable simultaneous acquisition of co- and cross-polarized optical images, two identical CCD cameras 402, 404 can be coupled via polarizing beam splitter 410. Fast and sensitive CCD cameras that can afford high spatial resolution are employed. Lateral resolution and field of view can be controlled by CCD macro-lenses with adjustable magnification. This allows for variable magnifications depending on the dimensions of the investigated area. Maximal field of view can be about 25 mm×25 mm with a lateral resolution not worse than 12 p.m.

The system uses computer controlled illumination, acquisition, and data processing. To obtain the polarization difference images (superficial images), the acquired co- and cross-polarized reflectance images can be processed using the following formula: _{18=1M} where I_{II} and 1₁ are the images of the remitted light polarized in the directions parallel and perpendicular to the polarization of incident light, respectively. Methods for optical image analysis can include those described in Yaroslaysky et al., Journal of Investigative Dermatology, 121(2), 259-266 (2003), the entire contents of which is incorporated herein by reference.

For image acquisition, software algorithms are used that integrate highspeed fully automated illumination control; acquisition of the two simultaneously registered optical co- and cross-polarized images; processing of the images; automated zoom into the multiple user-selected regions of interest (ROI); image storage; maintenance and easy access to the database of the images/subjects.

The imaging device can be calibrated using resolution and color targets, Spectralon™ reflectance standards with varying reflectivity, absorbing dye solutions with added scattering particles, and human tumor specimens. Illumination and acquisition settings can be selected to improve performance of the device.

Thus, the system does not use contrast agents but uses registered co- and cross-polarized image acquisition for continuous acquisition of optical images. One factor is that due to the discrepancies in the efficiencies of polarizing beam splitter and other optical components with respect to transmission of two orthogonal polarizations of light, throughput of two reflectance channels may vary. This results in different acquisition times of the two channels. The channels can be balanced by the introduction of the neutral density filters into the optical path.

A polarization sensitive optical imager can provide rapid image acquisition (−5-10 ms per frame); FOV of up to 2.5×2.5 cm; lateral resolution of 8-12 pm. The image acquisition and processing algorithms integrate automated illumination and acquisition control, registration and processing of the images, image storage and easy access to the database of the images/subjects.

Thus, the present invention provides a multimodal optical and terahertz imaging system, that uses a solid-state mixer-based 580 GHz transceiver for integration with the optical imager. The device is a low maintenance and provides room temperature operation with high signal-to-noise ratio and fast coherent detection. Two of the high-resolution imaging systems employ these transceivers.

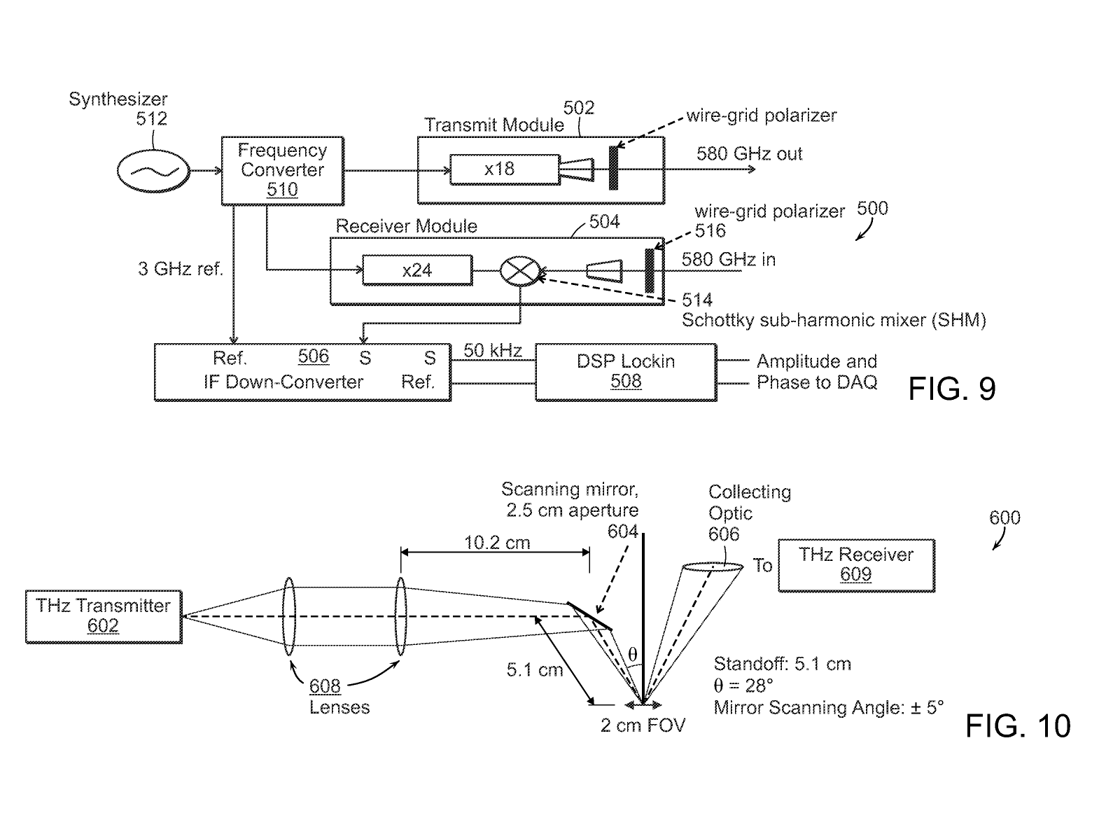

A 580 GHz frequency can be for illumination, since the contrast of cancer is maximal between 400-600 GHz. The transceiver 500 consists of six modules: the frequency synthesizer 512, the transmit multiplier chain 502, the receiver multiplier chain 504, the intermediate frequency (IF) converter 510, the I/Q demodulator 506 and the data acquisition hardware 508. In FIG. 9 a block diagram of the transceiver. The transceiver module for the frequency synthesizer 512 can generate three principal frequencies to drive the transmit multiplier and the receive multiplier chains, as well as for intermediate frequency (IF) phase reference. The synthesizer's center frequency can be shifted up by 62.5 MHz, resulting in a 3 GHz IF at the receiver after the multiplier chain (×48). It also provides a 3 GHz reference, which can be down converted in the IF chain.

The transmit multiplier chain can include an amplified quadrupler, followed by two varactor doublers and a tripler to achieve the ×48 multiplication factor. The tripler can be attached to a horn that will transmit the output signal. Wire grid polarizers 516 positioned in the output and return paths will have extinction ratio better than 10000:1. The system does not require a wide transmit frequency bandwidth. Therefore, a transmit beam power of 1 mW can be utilized.

For detection, a heterodyned Schottky sub-harmonic diode mixer 514 can be used. The local oscillator (LO) can be generated by converting the synthesizer signal in the same manner as the transmitter. The received signal can be mixed with the LO in a Schottky diode mixer, and down converted to the 3 GHz IF signal. Before entering the mixer a wire-grid polarizer can be used to select the cross-polarized component of the return signal. The IF converter amplifies and down converts the IF sample and reference signals to an appropriate frequency. The sample and reference signals can be passed to a lock-in amplifier to recover the amplitude and phase. The Noise Equivalent Power (NEP) of the receiver is 4×10⁻¹⁹ W/Hz. This receiver offers fast, room temperature, coherent detection. The transceiver can be integrated with an opto-mechanical scanning device. The commercially available 2D scanner consists of two galvanometric 25 mm aperture mirrors that can raster scan the beam across the sample at a rate of 2 frames per second or more.

The terahertz imager 600 is presented in FIG. 10. To enable seamless integration with the optical imager, off-axis scanning can be used. To focus the terahertz beam from source 602 on the sample two anti-reflection coated z-cut quartz lenses 608 are used. The beam waist at the focal plane is predicted to be 0.5 mm. The scanner 604 is placed between the second lens and the focal plane to allow the scanning mirrors to deflect the beam onto the sample with minimal distortion. The maximal deflection angles are selected so that the scanner never impinges on the field of view (FOV) of the optical system, while the scanned area is the same as the FOV of the optical imager. The reflected terahertz beam can be collected at the specular angle by lenses 606 and relayed to the terahertz receiver 609. These lenses account for the slight variation in specular angle over the focal plane. THz illumination at an oblique angle creates an elliptical focal spot at the sample plane. The ellipticity can vary as the cosine of the incident angle, thus the sample plane may not be uniformly illuminated. Calibration procedures can be used to account for illumination and collection differences across the image plane. The imaging plane (the scanning resolution is 0.1 mm while the beam waist at the focal plane is 0.5 mm) can be over-sampled. Thus if necessary, the system can use spotlight synthetic aperture techniques to improve the image.

The system can scan the 2 cm FOV at 2 frames per second, with a scanning resolution of 0.1 mm in both the axes. The lock-in time constant can be set to 2.5 ps. Assuming a source power of 1 mW, the projected SNR will be 83 dB. The sample reflection levels observed in our preliminary studies are approximately 24 dB below full scale. Thus the terahertz imager can have sufficient SNR to detect skin cancer. To improve image quality, amplitude and phase information can be used for implementing post processing noise reduction algorithms with data processor 204, such as DC bias subtraction. System calibration allows for quantitative terahertz reflectance imaging.

The selection of 580 GHz frequency is based on data acquired with far infrared gas laser systems, however these are not clinically useful. However, this operating frequency is between two systems used (524 GHz and 660 GHz). Moreover, as 524 GHz is within the optimal contrast range (400 GHz-600 GHZ) for cancer, a transceiver at 524 GHz can be used.

The system operates at 2 frames per second imaging or more. The imaging rate can be obtained with an opto-mechanical scanner using a heterodyne based detection system.

As the system does not require a wide transceiver bandwidth, the estimated output terahertz power is approximately 1 mW. This output power yields 83 dB of SNR. An output power of 1 mW can be uses, or alternating a 100 pW of output power can be used and accounting for the losses in the system, the SNR of the imager is about 73 dB, which is sufficient to detect skin cancer.

Stray reflections from system components may contaminate the resulting image. Range gate software can be used to eliminate the noise using a swept frequency finite bandwidth source. In particular, using a ramp sweep and an appropriate selection of sweep bandwidth, time gate spurious signals and increase the system SNR to 100 dB for a 100 pW source at the expense of decreased frame rate. The estimates show that in the worst case, scan time per frame increases to 4 seconds.

The terahertz camera module does not require mechanical scanning device. The SNR of an imager with output source power of 1 mW, which corresponds to the power density of 2.5×10⁻⁶ W/MM² over a 20 mm×20 mm FOV. Given a 100 μm×100 μm pixel size yielding a maximum power of 2.5×10⁻⁶ W per pixel. For the frequency range between 400 and 600 GHz, the best available THz camera module is a CMOS focal plane array, which demonstrated a minimum observed NEP of 300×10⁻¹² W/′iHz at 650 GHz. Thus, with 1 mW of output power, without losses, and 4 s integration time, the imager yields a SNR of 22 dB, which is prohibitively lower than SNR required for cross-polarized terahertz reflectance imaging of skin.

A reflectance polarization sensitive 580 GHz imaging system can have a 0.6 mm spatial resolution and 2 cm field of view with a signal to noise ratio better than 70 dB. The system is capable of generating images at a rate 2-0.25 frames/second.

The optical and terahertz systems can be integrated into a single unit, with common imaging plane, image acquisition and hardware control. The main advantage provided by combining these two imaging modalities into one imaging device is its ability to rapidly acquire registered optical and terahertz images. Thus, the time required for the detection of tumor margins can be dramatically reduced as compared to using the two separate units. At the same time, the accuracy of tissue discrimination can be significantly increased, as continuous wave cross-polarized terahertz images can macroscopically identify tumor nests, registered polarized light optical images enable higher resolution inspection of tissue morphological changes within the suspicious areas identified by terahertz imaging.

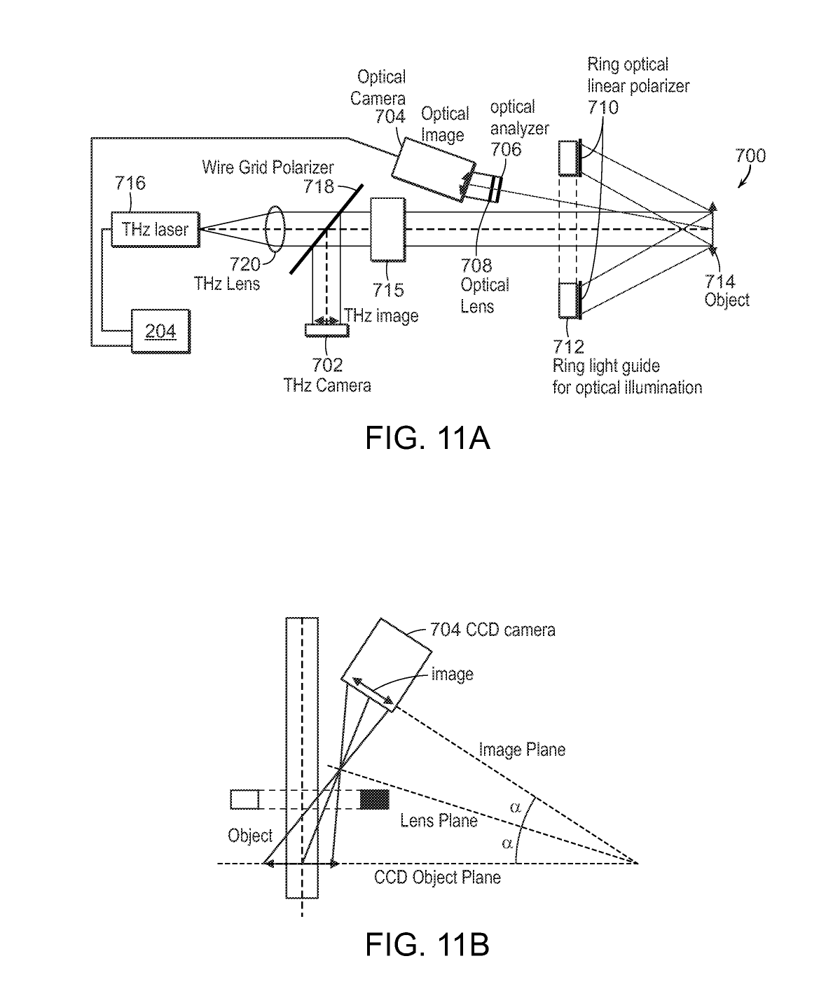

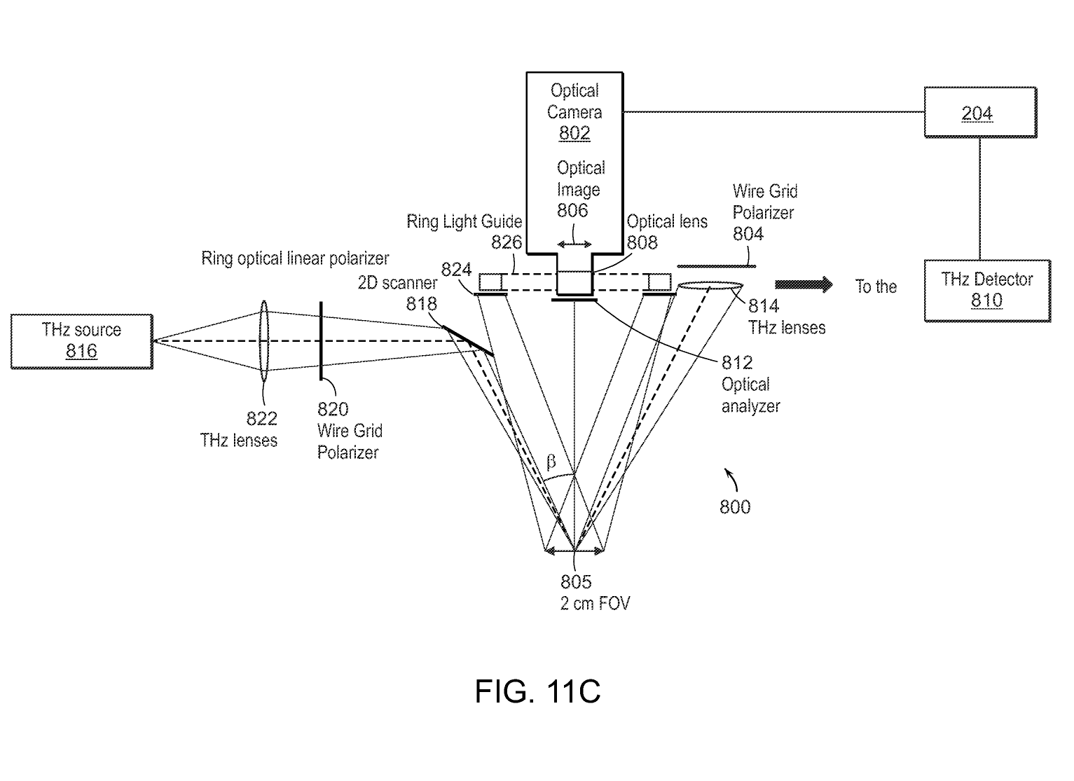

Preferred embodiments of the system 700 are presented in FIGS. 11A-11C. For optical imaging, a LED ring light source 712 can be used for illumination. The wavelength of this source is be between 395-470 nm. The light incident on the sample 714 can be linearly polarized through a ring linear polarizer 710 optimized for the illumination wavelength. The light remitted from the sample passes through the objective lens 708 and split by the polarizing beam splitter 706 into two orthogonal polarizations (co- and cross-polarized with respect the incident beam). The co- and cross-polarized optical signal are simultaneously captured by two identical CCD cameras 704. For terahertz imaging, the beam output from the source 716 is focused on the imaging plane (object plane) by a system of terahertz lenses 720. The beam is linearly polarized using a wire grid polarizer 718. Optionally, two galvanometric mirrors can be used to scan the imaged point or light spot in x and y directions over the imaged plane. The 2D galvanometric scanner 715 will reflect the beam onto the sample plane at an off-axis angle. The angle will be selected for the scanner never to limit the field of view (FOV) of the optical system, while the scanned area will completely overlap the FOV of the optical camera. The reflected terahertz beam from the sample is collected by lenses and sent through another wire-grid polarizer which will transmit the cross-polarized component (or co-polarized component) to the terahertz receiver 702.

The system terahertz and optical image acquisition, control, and processing software to enable highspeed fully automated illumination control; acquisition of the simultaneously registered optical co- and cross-polarized images and cross-polarized terahertz image; processing of the images; automated zoom into the multiple user-selected regions of interest (ROI) in the optical images, as well as automated zoom into the optical images within the areas of decreased terahertz reflectivity; image storage; maintenance and easy access to the database of the images/subjects is accomplished using data processor 204. The combined imaging device can be used to measure resolution, color targets, tissue phantoms, and human tumor specimens.

The system 800 of FIG. 11C shows the terahertz source 816, lens 822, wire grid polarizer 820, scanner 818, terahertz detectors 810 coupled through polarizer 804 and lens 814 to the sample region 805. The optical source 826 and polarizer 824 illuminate the sample region 805 to detect images with detectors 802 using analyzer 812, and lens 808 to form the image 806 at the detection surface. The processor 204 processes the image data as described herein.

The acquisition time of the optical images can be much shorter than that of terahertz images. The terahertz images can be acquired at 0.25 frames per second or more, for example. The optical images can be acquired at 5-10 msec per frame. To avoid impact of the artifacts caused by object movement, the system can continuously acquire optical images during terahertz acquisition to track those artifacts and reject the frames, affected by the movement from the analysis.

A multimodal polarization sensitive optical and terahertz imager will be constructed and tested using resolution targets, phantoms, and tissue specimens. An optical component of the imager will provide rapid image acquisition (−5-10 ms per frame); FOV of up to 2.5×2.5 cm; lateral resolution of 8-12 μm. A terahertz component of the imager can provide 580 GHz illumination, a 0.6 mm spatial resolution and 2 cm field of view with a signal to noise ratio better than 70 dB. The system is capable of generating images at arate 2-0.25 frames/second. The hardware control and image acquisition algorithms integrate the following functions: automated illumination and acquisition control, registration and processing of the images, image storage and access to the database of the images/subjects.

The data base of multimodal optical and terahertz images of normal and pathological skin structures is collected, compared side by side with histopathology, and analyzed.

The data collection and analysis algorithm is as follows. 1) Registered optical and terahertz images are acquired; 2) For the analysis, the registered optical and terahertz images are overlaid or fused; 3) En face frozen H&E histopathological sections are processed from the imaged piece of tissue; 4) Terahertz images are quantified and the reflectivity values corresponding to different skin structures are determined from the optical images. The appearance of the tissue structures in the optical images can be verified by comparison to histopathology; 5) The databases of optical images with corresponding terahertz reflectivity values of healthy (i.e., collagen, hair follicles, sebaceous glands, eccrine glands, nerves, etc.) and pathological (i.e., cancer, actinic keratosis, inflammatory infiltrate, etc.) are stored and used as a reference in the course of the subsequent measurements; 6) Phenomenological threshold values of terahertz reflectivity for cancer tissue are used in the course of subsequent measurements.

Morphological features and appearance of different tissue structures in the optical images is evaluated and compared with corresponding histopathology. The values of terahertz reflectivity, corresponding to different tissue structures are determined. The differences of terahertz reflectivity of pathological (i.e., cancer, actinic keratosis, inflammatory infiltrate, etc.) and normal structures (such as collagen, hair follicles, sebaceous glands, etc.) can be calculated and used for tissue differentiation in the course of subsequent measurements. The databases of optical images with corresponding terahertz reflectivity values of healthy and pathological tissues are stored and used as a reference in the course of the subsequent measurements. The differences of terahertz reflectivity in cancerous and normal structures will be determined and statistically analyzed using a paired Student t test.

To estimate the sample size, statistical power calculations have been performed. Data show that at least 10 samples are necessary in order to have 98% statistical power to detect differences in the terahertz reflectivity values between normal and cancerous tissue. Generally, the normal skin exhibits average terahertz reflectivity of approximately (8.40±0.1)×10-3 and cancerous skin of (6.90±0.34)×10-3. To increase the probability that required characteristics will be met, at least 11 samples of each subtype of NMSC are used for analysis.

In correlating optical images with histopathology the system uses similarities in the morphology and visual appearance of tissue structures. In practice, due to the preparation technique of the frozen section, it may be stretched or shrunk in comparison with the remaining thick piece of skin. To reduce the influence of these artifacts the system digitizes the histological slides and identifies four to ten pairs of common features in histology and in the optical images. Then overlay optical and histopathological images by applying affine transformations. These procedures enable comparison of the optical and terahertz images to the corresponding histopathology.

The data base of optical images of normal, pathological, and cancerous tissues with corresponding terahertz reflectivity values can be collected. Threshold values of terahertz reflectivity for cancer tissue can be determined.

The optical and terahertz polarization images are evaluated by comparison to the en face frozen H&E histopathological sections, processed from the imaged tissue. In total, we will image 60 samples, including 50 from skin excisions positive for NMSC (10 nodular BCCs, 10 infiltrative BCCs, 10 superficial BCCs, 10 invasive SCCs and 10 SCCs in situ) and 10 samples negative for NMSC as controls.

For the in vitro measurements, viable tumor material can be collected within forty minutes after excision from Mohs micrographic surgeries can be used. The specimens are rinsed, photographed, and imaged. Registered terahertz and optical images are acquired. Terahertz images are quantified based on the terahertz reflectivity values and confirmed by the analysis of tissue morphology from optical images the tumor nests are detected and the tumor margins are outlined. The results can be evaluated by comparison to en face frozen histopathology. In total, images of 50 samples positive for nonmelanoma skin cancers and 10 samples negative for NMSC can be used as controls. The regions of terahertz images with reflectivity lower than that of the established cancer threshold can be identified. Optical images are inspected and diagnosed using the data base of images collected.

For evaluation, the digitized histology slides can be compared to the resulting optical, terahertz and multimodal images of thick fresh skin excisions. In practice, due to the processing artifacts of histology, frozen sections can be stretched or shrunk in comparison with the imaged thick tissues. To reduce the influence of these artifacts on the comparison, four to ten pairs of common features can be labeled in the digitized histology slides and in the images. Overlaying the obtained images with histopathological images by applying affine transformations. The accuracy of the transformations can be checked by applying the algorithm for correcting the distorted image of the known object. The following criteria for the comparison of images to corresponding histopathology. Similarity in the location of the tumor in histology and the images can serve as the first criterion. To quantify the accuracy of the technique, the surface areas occupied by the tumor in the images (S,) and histological slides (Sh) are be processed and compared. The ratio of the cancerous areas in the image and histology serves as the second criterion. The agreement can be considered acceptable if the tumor area in the image equals or up to 10% greater than that in histology Si/Sh<1.1), i.e. would corresponds to complete tumor removal by image-guided surgery. The contrast of the lesion with respect to the surrounding healthy tissue in the terahertz images can be used as the third criterion. The contrast of the cancerous and normal skin in the images can be calculated by averaging the terahertz reflectivity values over the entire cancer or normal areas, respectively. The contrast of a lesion with respect to the surrounding normal skin, CAN, can be evaluated as the difference of the average terahertz reflectivity value in the tumor and in the healthy skin of the respective image multiplied by 100. The threshold for contrast values can be chosen to guarantee that the difference of the cancerous and normal averaged terahertz reflectivity value is significantly (at least 10 times) greater than the noise level.

First compare the results for the binary indicator of absence or presence of a tumor (similarity in location). The Receiver Operating Characteristic (ROC) and associated parameters, i.e., sensitivity, specificity, positive predictive value, negative predictive value, will be calculated to determine the level of accuracy of the optical imaging, terahertz imaging, and multimodal imaging against the standard of histopathology. The results of the multimodal imaging is useful if the terahertz image correctly identifies location of the tumor while the optical image correctly identifies the extent of the tumor. Descriptive statistics can be provided for the contrast of the lesion with respect to the surrounding healthy tissue in the terahertz images and confidence intervals will be provided for this criterion. In addition, descriptive statistics can be provided for measurement criteria by tumor subtype.

Note that the frozen section histology exhibits folds or tissue loss as compared to the thick imaged sample. In order to correct for this, multiple 5 micron thick sections can be cut from the imaged tissue block and the most appropriate section can be digitized for comparison.

The device can be used as an intraoperative tool for identifying squamous cell carcinoma in mice. Malignant squamous cell carcinoma (SCC) can be imaged in live mice. For the measurements SENCAR mice are used. SENCAR stands for SENsitivity to CARcinogenesis. These mice have been used extensively for skin carcinogenesis measurements. The resulting optical and terahertz images can be compared to the en face hematoxylin and eosin (H&E) histopathological sections processed from the imaged tissue. Reflectivity values for terahertz cross-polarized reflectance images can be quantified. The morphological features in the optical images can be identified. The sensitivity and specificity of the developed technique can be determined based on comparison of the imaging results to the diagnosis based on the analysis of the H&E histopathology.

Firstly, the terahertz reflectivity thresholds for the SCC in mice can be used. Measurements show that at least 10 samples can be used in order to have 98% statistical power to detect differences in the terahertz reflectivity values between normal and cancerous tissue. Based on the results obtained for human skin, normal mouse skin exhibits average terahertz reflectivity of approximately (8.40±0.1)×10-3 and cancerous skin of (6.90±0.34)×10-3.

The backs of the mice can be shaved and treated with a single application of DMBA (20 pg in 200 pl of acetone) and followed a week later by twice weekly applications of DMBA for 17-20 weeks. The number and size of lesions on each mouse can be recorded every week. After 20 weeks of the treatment, multiple SCCs occur in 100% of mice. 3°-33 Mice will be sacrificed if they are moribund or following imaging of carcinomas.

Before imaging the mice can be shaved and anesthetized. Intraperitonial anesthesia (Ketamine 90 mg/mI and xylazine 10 mg/mI, mixed) 40 pl/mouse/dose can be injected using insulin syringe (28G). Five (5) minutes following anesthesia, the tumors are excised. The surgical bed and the fresh cut surface of the excision can be imaged for the assessment of the entire tumor margin in vivo and ex vivo, respectively. The imaged lesions can then be excised, fixed in formalin, processed and stained with hematoxylin and eosin (H&E) for histological examination.

Morphological features and appearance of different tissue structures in the optical images will be evaluated and compared with corresponding histopathology. The values of terahertz reflectivity, corresponding to normal and cancerous tissue structures will be determined. The databases of optical images with corresponding terahertz reflectivity values of healthy and cancerous mouse tissue can be created and stored to be used as a reference in the course of the subsequent trial. The differences of terahertz reflectivity in cancerous and normal structures can be determined and statistically analyzed using a paired Student t test.

In vivo mouse skin optical and terahertz polarization images will be evaluated blindly by comparison to the en face frozen H&E histopathological sections, processed from the imaged tissue. Thirty SENCAR mice (>18 g body weight) male and female can be used to measure the required characteristics. The mice can be divided into two groups: group 1 and 2. Malignant squamous cell carcinoma will be initiated and promoted in mice from group 1. Mice from group 2 will not be treated (reference group).

SCC can be induce in mice from group 1 following the same procedure described herein. The mice from the reference group 2 are not treated. Before imaging the mice can be shaved and anesthetized. Intraperitonial anesthesia (Ketamine 90 mg/ml and xylazine 10 mg/mi, mixed) 40 pL/mouse/dose is injected using insulin syringe (28G). Five (5) minutes following anesthesia, the tumors with adjacent skin are excised. The surgical bed and the fresh cut surface of the excision can be imaged for the assessment of the entire tumor margin in vivo and ex vivo, respectively. The imaged skin and lesions are excised, fixed in formalin, processed and stained with hematoxylin and eosin (H&E) for histological examination. Image 30 of mice in vivo (15 mice per group) with at least 15 SCC lesions, as well as 30 mouse skin excisions with at least 15 SCC tumors can be used.

The regions of terahertz images with reflectivity lower than that of the established cancer threshold are identified. Optical images are inspected and diagnosed using the data base of images collected during the animal measurements in the manner similar to that of histopathology. As morphological features and appearance of different tissue structures in the polarization optical images is similar to those in histopathology.

Digitized histology slides can be compared to the resulting in vivo and ex vivo mouse optical, terahertz and multimodal images. The following criteria for the comparison of images to corresponding histopathology. Similarity in the location of the tumor in histology and the images will serve as the first criterion. To quantify the accuracy of the technique, the surface areas occupied by the tumor in the images (Si) and histological slides (Sh) can be processed and compared. The ratio of the cancerous areas in the image and histology can serve as the second criterion. The agreement will be considered acceptable if the tumor area in the image will be equal or up to 10% greater than that in histology (1 . . . SiSh<1.1), i.e. would correspond to complete tumor removal by image-guided surgery. The contrast of the lesion with respect to the surrounding healthy tissue in the terahertz images can be used as the third criterion. The contrast of the cancerous and normal skin in the images can be calculated by averaging the terahertz reflectivity values over the entire cancer or normal areas, respectively. The contrast of a lesion with respect to the surrounding normal skin, can be evaluated as the difference of the average terahertz reflectivity value in the tumor and in the healthy skin of the respective image multiplied by 100. The threshold for contrast values can be chosen to guarantee that the difference of the cancerous and normal averaged terahertz reflectivity value is significantly (at least 10 times) greater than the noise level.

The results for the binary indicator of absence or presence of a tumor (similarity in location) can be analyzed. The Receiver Operating Characteristic (ROC) and associated parameters, i.e., sensitivity, specificity, positive predictive value, negative predictive value, will be calculated to determine the level of accuracy of the optical imaging, terahertz imaging, and multimodal imaging against the gold standard of histopathology. The results of the multimodal imaging are useful, for example, if the terahertz image correctly identifies location of the tumor while the optical image correctly identifies the extent of the tumor. Descriptive statistics can be provided for the contrast of the lesion with respect to the surrounding healthy tissue in the terahertz images and confidence intervals can be provided for this criterion. In addition, descriptive statistics can be provided for measurement criteria by tumor subtype.

It is possible that the frozen section histology exhibits folds or tissue loss as compared to the thick imaged sample. In order to correct for this multiple, 5 micron thick sections can be cut from the imaged tissue block and the most appropriate section is digitized for comparison. Ex vivo and in vivo images of the same lesion will exhibit differences. These differences can be documented and analyzed. Independent analysis of the in vivo and ex vivo sets of images or side-by-side analysis of these two sets of images can be performed. This comparison provides a basis for correlating in vitro and in vivo appearance of skin structures.

Blood in the imaging field may present a problem for the quality of in vivo imaging. To control bleeding after excision, aluminum chloride (AICl3) in 20% solution can be applied if needed. AICl3 is used conventionally as a hemostatic agent for superficial wounds. After AICl3 administration and establishment of hemostasis, the wound can be rinsed with sterile water and imaged. Preliminary results demonstrate that AICl3 does not affect the quality of optical images of skin adversely.

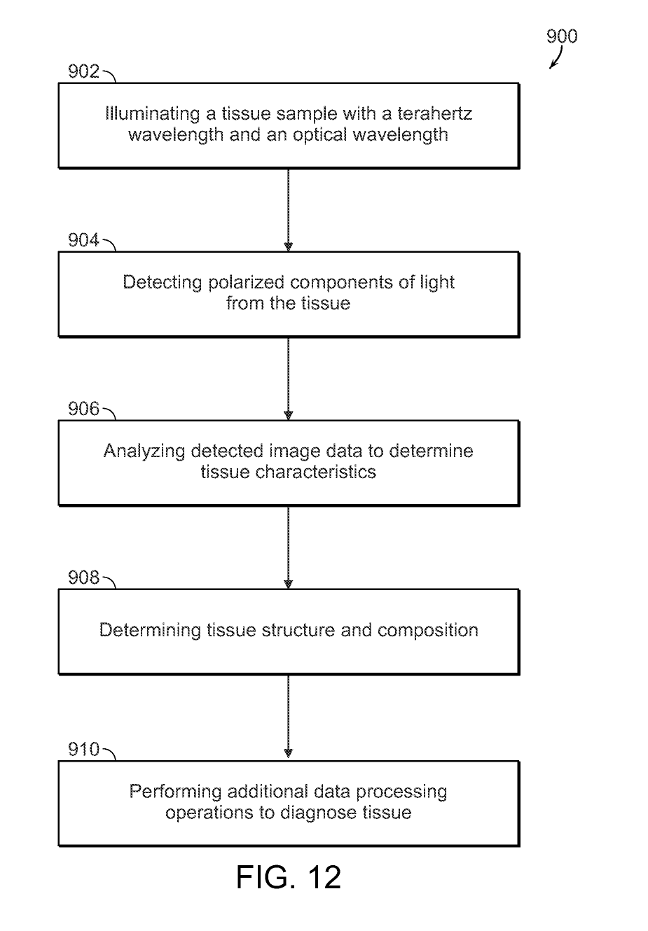

A method of imaging in accordance with the invention is shown in the process sequence of FIG. 12. Method can include methods of analysis described by Woodard et al., in the Journal of Biological Physics 29:257-261 (2003), the entire contents of which is incorporated herein by reference. This method employs Fourier transformation of the image data to the frequency domain and can also utilize time domain processing in which depth information can be obtained from time post pulse (TPP) analysis. The method 900 combines optical and terahertz imaging of a sample as described herein. In this method, a tissue sample is illuminated 902 with terahertz and optical wavelengths of light. The detected polarized components 904 are then processed by a data processor to analyze 906 the detected image data. The processed image data can be used to determine 908 tissue structure and composition. Additional data processing 910 operations based on frequency domain and/or time domain processing can also provide diagnostic information for the detection of cancer.

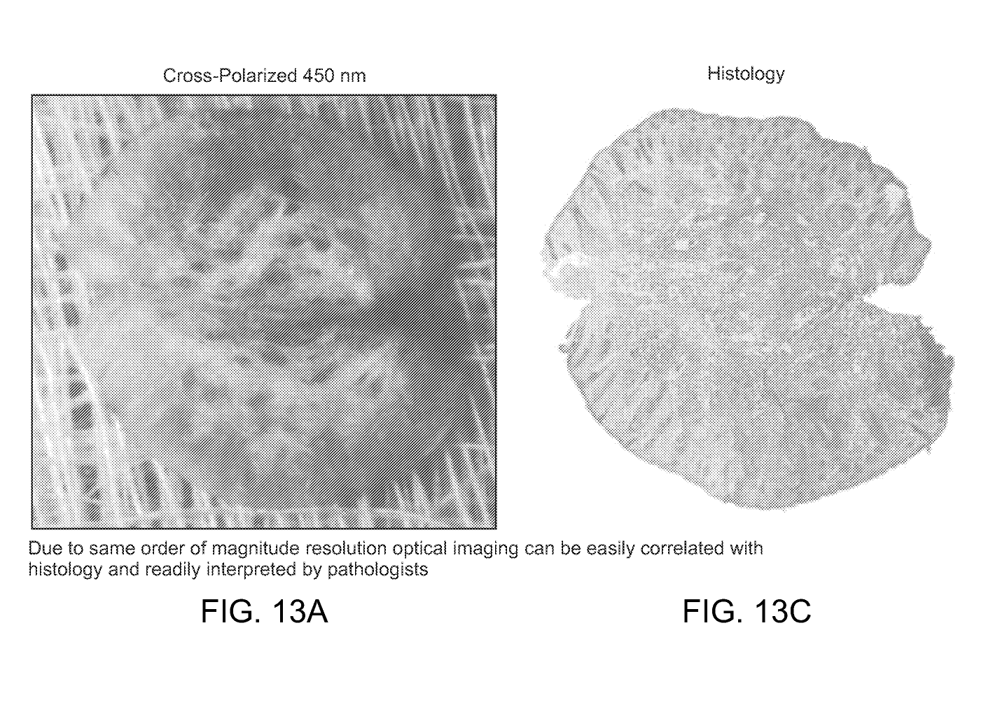

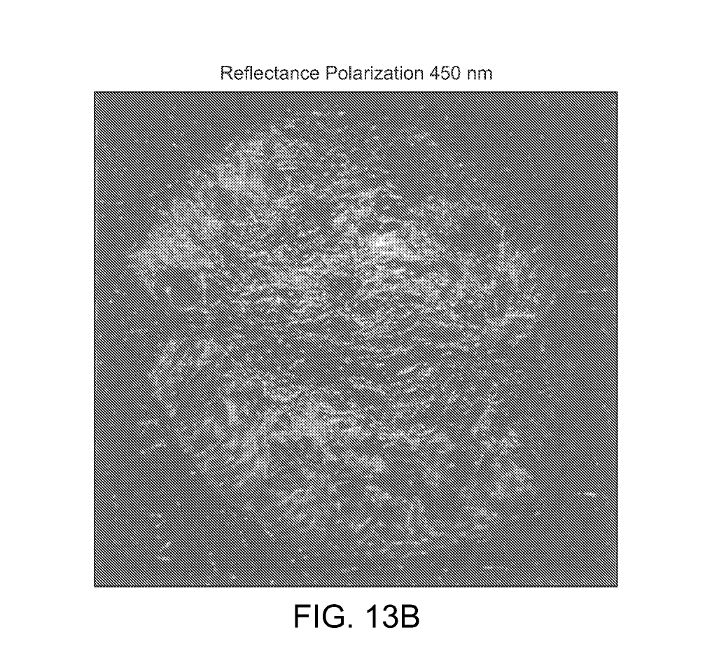

FIGS. 13A-13C show comparative data of tissue samples using optical imaging using cross-polarized imaging at 450 nm, reflectance polarization imaging at 450 nm and a standard histology image.

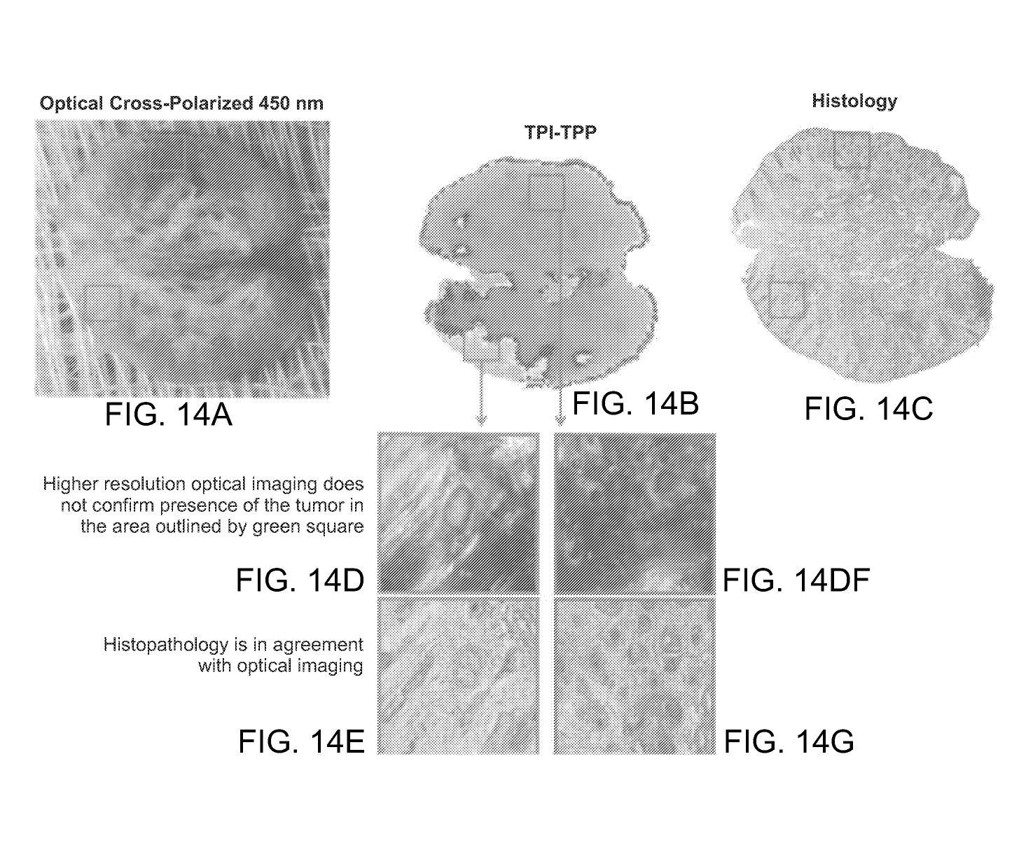

FIGS. 14A-14G illustrate the use of comparative high resolution imaging of tissue samples using optical and terahertz imaging techniques as described herein. FIG. 14A shows upper and lower regions of interest (ROI) in an optical cross-polarized image. FIG. 14B shows a terahertz image with the same ROIs marked, and FIG. 14C shows the same ROIs marked in an histology image. The detailed ROI analysis of this sample confirms that no tumor is present based on the cross-polarized images at 450 nm (FIGS. 14D and 14F) and the histology images (FIGS. 14E and 14G).

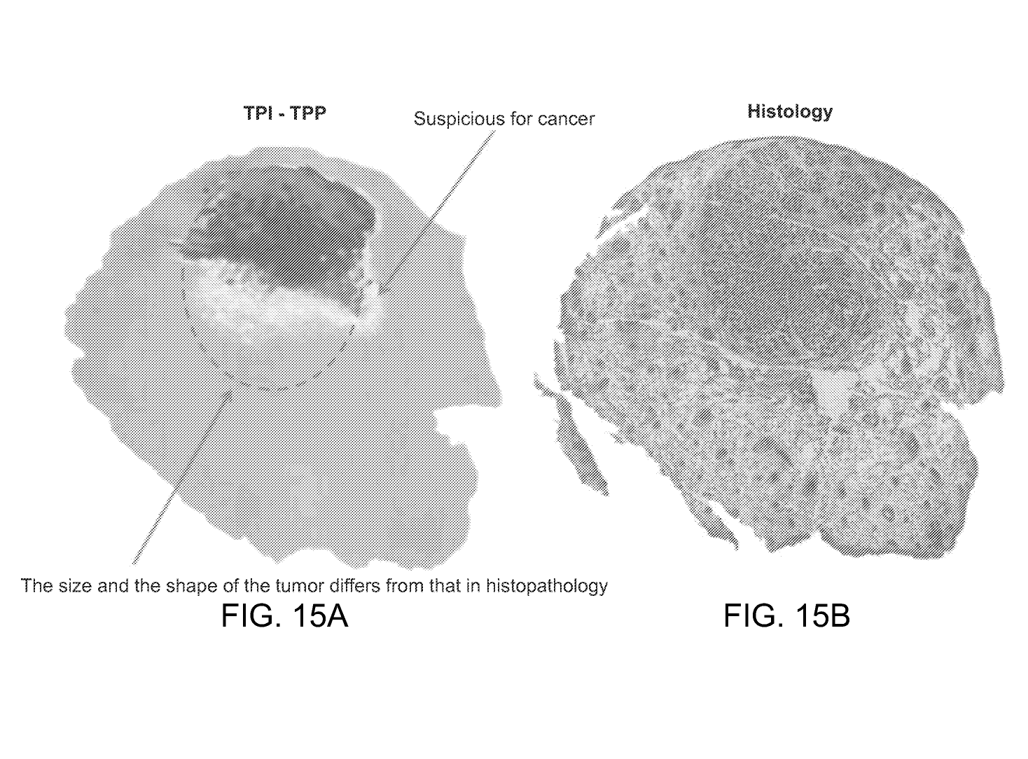

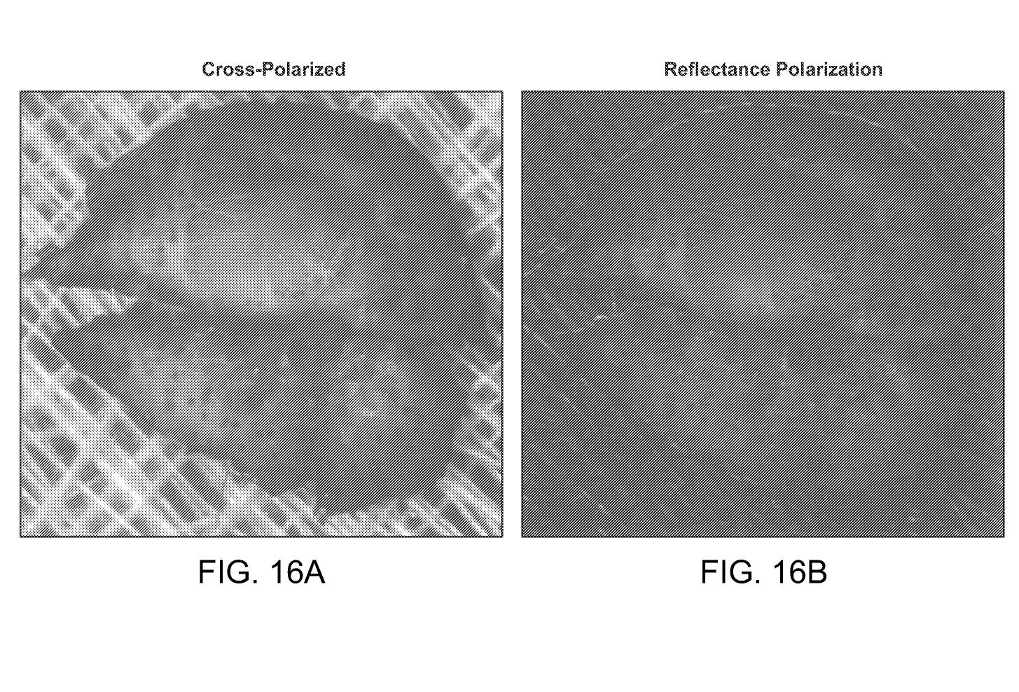

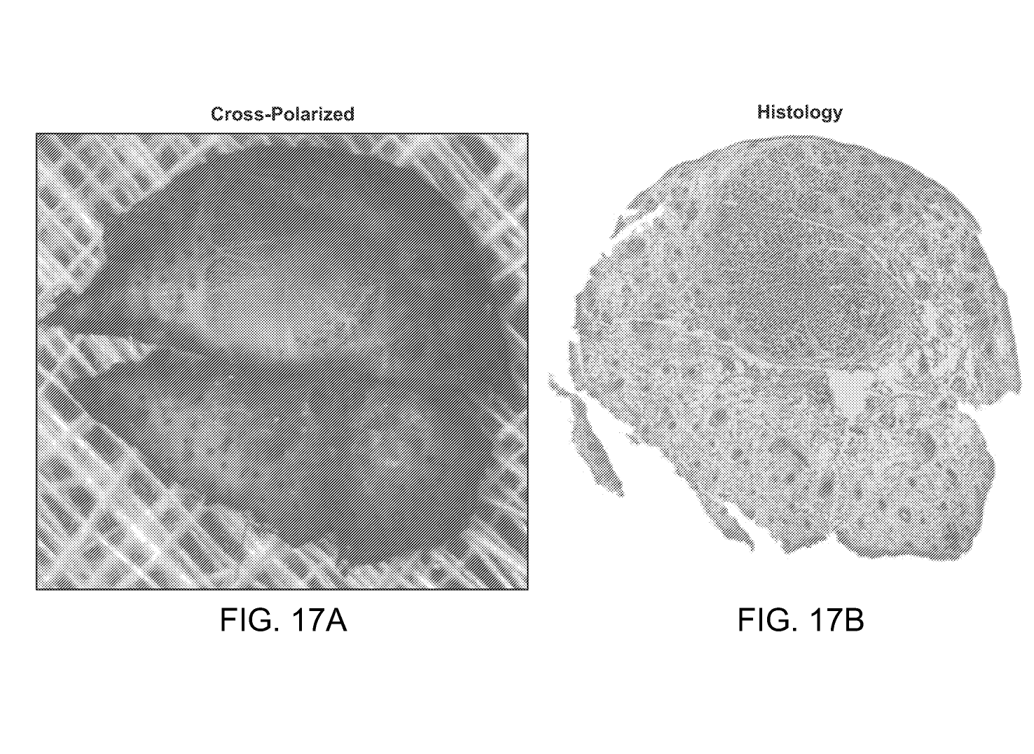

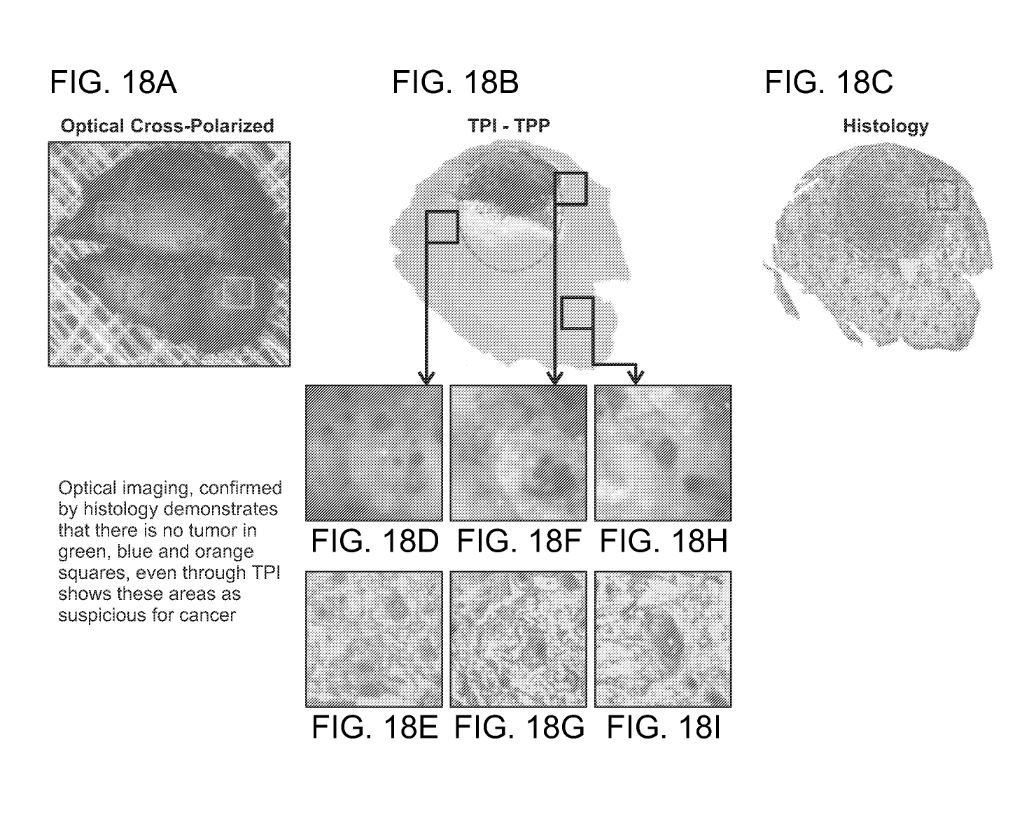

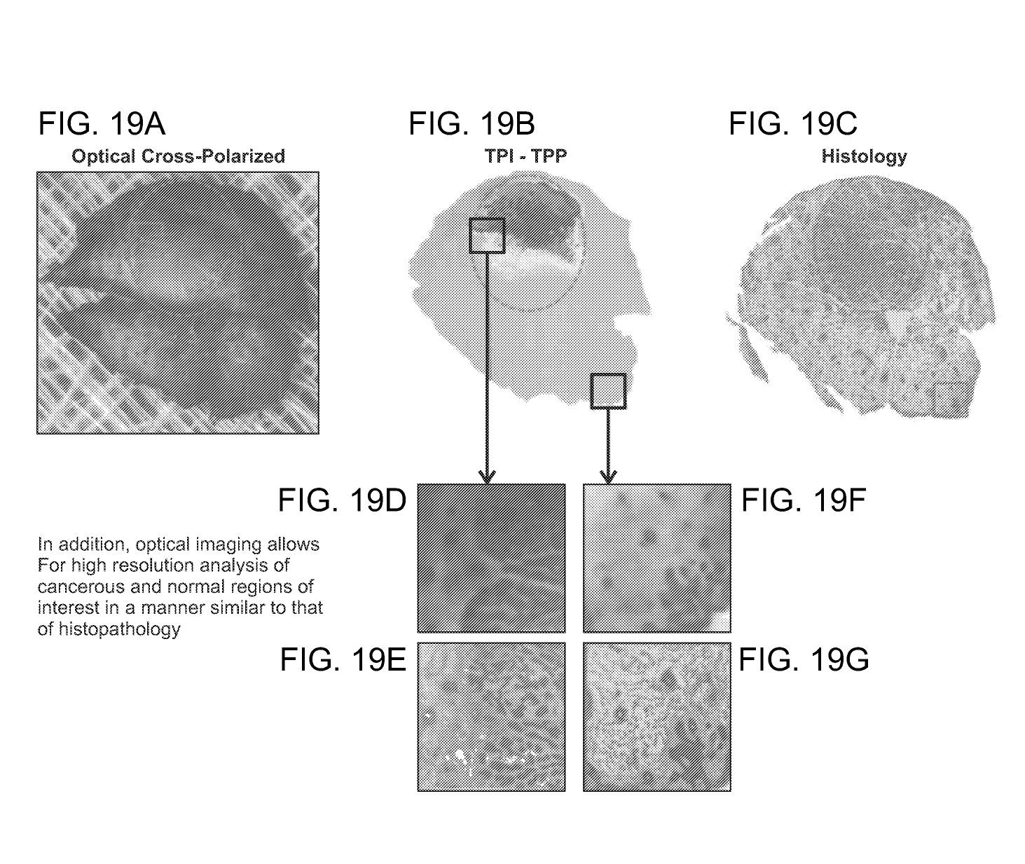

FIGS. 15A and 15B illustrate comparative images using terahertz and histology images to resolve tumor shape in a tissue sample. FIGS. 16A and 16B compare cross-polarized and reflectance polarized images of the tissue sample. FIGS. 17A and 17B compare the cross-polarized and histology images of the sample. Shown in FIGS. 18A-18I are images that compare the indicated ROIs in the cross-polarized, terahertz and histology images which confirm no tumor tissue is contained within these regions. FIGS. 19A-19G analyze additional regions which confirm cancer in the upper region (FIGS. 19D and 19E) and confirm normal tissue in the lower ROI (FIGS. 19F and 19G).

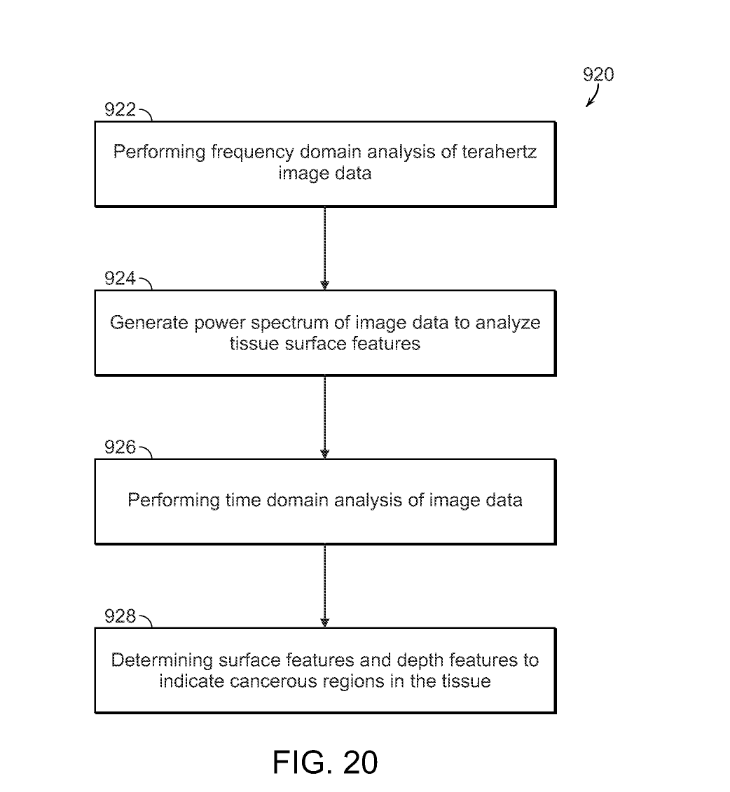

Shown in FIG. 20 is a process sequence in which terahertz image processing methods are illustrated in accordance with preferred embodiments of the invention. The processing can include the use of Fourier transformation of image data to the frequency domain 922 which can provide power spectral data 924 useful in the identification of surface features. Time domain analysis 926 can also be used to indicate both surface and sub-surface features to diagnose tissue.

The claims should not be read as limited to the described order or elements unless stated, all embodiments that came within the scope and spirit of the following claims and equivalents thereto are claimed as the invention.

1. A method for measuring tissue comprising:
illuminating a region of tissue with light from a first light source, the illuminating light having a terahertz wavelength;
detecting a first polarization component of light from the region of tissue and a second polarization component of light from the region of tissue; and
processing the first detected polarization component and the second detected polarization component to determine a characteristic of tissue.

2. The method of claim 1 further comprising forming an image of the region of tissue wherein the processing step of determining a characteristic of the tissue comprises determining a structural characteristic.

3. The method of claim 1 further comprising processing spectral data with a data processer and wherein the processing step further comprises determining molecular composition of the tissue.

4. The method of claim 1 further comprising illuminating the tissue with light from a second broadband light source within an optical wavelength range.

5. The method of claim 1 further comprising performing cross-polarized imaging with light reflected by the tissue.

6. The method of claim 4 further comprising detecting light from the region of tissue in response to illuminating light from the second light source, the detected light including a first polarization component detected with a first detector and a second polarization component detected with a second detector.

7. (canceled)

8. The method of claim 1 further comprising illuminating the tissue with a continuous wave terahertz source and detecting light with an optical detector and a terahertz detector.

9. (canceled)

10. (canceled)

11. The method of claim 4 further comprising illuminating the tissue with a ring illuminator.

12. The method of claim 1 further comprising scanning a beam across a tissue surface.

13. The method of claim 1 further comprising processing a terahertz image with a frequency domain representation of the image.

14. The method of claim 1 further comprising detecting a plurality of temporally sequenced images and performing time domain processing of the plurality of images.

15. The method of claim 1 further comprising comparing a region of interest of an optical image with the same region of interest in a terahertz image.

16. The method of claim 1 further comprising comparing a detected image with a histological image of the sample.

17. (canceled)

18. The method of claim 1 further comprising illuminating the tissue with polarized light and transmitting light from the tissue through a polarizer.

19. (canceled)

20. A multimodal system for imaging tissue comprising:
a first light source that generates a terahertz wavelength of light;
a second light source that generates an optical wavelength of light;
a light coupling system that couples light from the first light source and the second light source onto a region of tissue;
a first detector that detects light from the region of tissue in response to light from the first light source; and
a second detector that detects light from the region of tissue in response to light from the second light source.

21. The system of claim 20 wherein the first detector detects a first polarization component and a second polarization component.

22. The system of claim 20 further comprising a polarizer positioned to couple light from the tissue to the second detector and wherein the second detector detects a first polarization component and a second polarization component.

23. The system of claim 20 further comprising a data processor connected to the first detector and the second detector, the data processor being programmed to process image data to determine a structural characteristic of the tissue and a concentration of a molecular component of the tissue.

24. (canceled)

25. The system of claim 20 wherein the first light source is a continuous wave laser light source.

26. (canceled)

27. The system of claim 20 further comprising a ring illuminator.

28. The system of claim 20 further comprising a scanner to scan light from a light source across a tissue surface.

29. The system of claim 20 further comprising a polarizer that polarizes light incident on the tissue and an analyzer to select a polarization component.

30. (canceled)

31. The system of claim 20 further comprising a light delivery lens system to illuminate the tissue and a light collection lens system to couple light from the tissue to a detector.

32. (canceled)

33. The system of claim 20 further comprising a terahertz lens and an optical lens.

34. The system of claim 20 further comprising a terahertz transceiver.

35. The system of claim 20 further comprising a terahertz transmitter module and a terahertz receiver module.

36. The system of claim 20 further comprising a terahertz receiver module.

37. The system of claim 20 further comprising a frequency converter and a down converter.

38. The system of claim 23 wherein the data processor identifies a plurality of tissue components such as collagen, fat, tumor, epidermis and/or Pilo-sebaceous complex.

39. (canceled)

40. (canceled)
